(19) United States
(12) Patent Application Publication
(10) Pub. No.: US 2024/0197687 A1
(43) Pub. Date: Jun. 20, 2024

(54) METHODS OF TREATING NEUROINFLAMMATION AND INHIBITING MONOAMINE OXIDASE-A AND RESTORING/UPREGULATING/INCREASING IL-13 AND AND PGC-1 BY ADMINISTERING ORAL DOSAGE FORM CONTAINING MICRONIZED AND NONMICRONIZED PARTICULATE METAXALONE, ALGINIC ACID AND PROPYLENE GLYCOL ALGINATE

(71) Applicant: Primus Pharmaceuticals, Inc., Scottsdale, AZ (US)

(72) Inventors: Francesco Squadrito, Messina (IT); James C. Lukban, Scottsdale, AZ (US); Ryan E. Hartung, Scottsdale, AZ (US); Garrett A. Campbell, Scottsdale, AZ (US)

(73) Assignee: Primus Pharmaceuticals, Inc., Scottsdale, AZ (US)

(21) Appl. No.: 18/215,170

(22) Filed: Jun. 28, 2023

Related U.S. Application Data
(60) Provisional application No. 63/390,563, filed on Jul. 19, 2022.
(60) Provisional application No. 63/358,104, filed on Jul. 2, 2022.

(51) Int. Cl. A61K 31/421; A61K 9/20; A61P 25/02; A61P 29/00

(52) U.S. Cl. CPC A61K 31/421; A61K 9/2018; A61P 25/02; A61P 29/00

(57) ABSTRACT

A method of neuroinflammation and conditions associated therewith including chronic and acute conditions as well as specific and non-specific conditions. Oral dosage forms containing 640 mg micronized and non-micronized particulate metaxalone and excipients including alginic acid and propylene glycol alginate. The method includes inhibiting the monoamine oxidase A (MAO-A) enzyme as well as restoring/upregulating/increasing IL-13 and PGC-1α. The treated conditions, among others, include diabetic neuropathy, fibromyalgia, back pain, chronic pain induced depression, restless leg syndrome, anxiety, mood disorder, peripheral neuropathy, and herpetic neuralgia. The method further includes inducing or restoring homeostasis, restoring oxidative homeostasis, restoring basal phenotype from a neuroinflammatory state and improving neuroplasticity.

### TECHNICAL FIELD

The technical field of the invention concerns small molecule pharmaceuticals used to effect inhibition of enzymes and enzymatic pathways and regulation of neurotrophic factors to treat inflammation of nerve tissue or neuroinflammation as well as treating brain white matter, demyelination diseases, sciatica, fibromyalgia, neuropathic pain, ischemic stroke and traumatic brain injury.

### CROSS-REFERENCE TO RELATED APPLICATIONS

This application claims priority to U.S. provisional application nos. 63/358,104, filed Jul. 2, 2022, and 63/390,563, filed Jul. 19, 2022, the contents of which are incorporated by reference as if fully set forth herein. Further incorporated herein by reference is the manufacturing and formulation subject matter of U.S. utility application Ser. No. 18/198,266, filed May 16, 2023.

### STATEMENT REGARDING FEDERALLY SPONSORED RESEARCH OR DEVELOPMENT

No federally-sponsored research or development.

### BACKGROUND OF THE INVENTION

Neuroinflammation. Neuroinflammation is inflammation of the nervous tissue. It may be initiated in response to a variety of cues, including infection, traumatic brain injury, toxic metabolites, or autoimmunity. In the central nervous system (CNS), including the brain and spinal cord, microglia are the resident innate immune cells that are activated in response to these cues. The CNS is typically an immunologically privileged site because peripheral immune cells are generally blocked by the blood-brain barrier (BBB), a specialized structure composed of astrocytes and endothelial cells. However, circulating peripheral immune cells may surpass a compromised BBB and encounter neurons and glial cells expressing major histocompatibility complex molecules, perpetuating the immune response. Although the response is initiated to protect the central nervous system from the infectious agent, the effect may be toxic and widespread inflammation as well as further migration of leukocytes through the blood-brain barrier.

Neuroinflammation is widely regarded as chronic, as opposed to acute, inflammation of the central nervous system. Acute inflammation usually follows injury to the central nervous system immediately, and is characterized by inflammatory molecules, endothelial cell activation, platelet deposition, and tissue edema. Chronic inflammation is the sustained activation of glial cells and recruitment of other immune cells into the brain. It is chronic inflammation that is typically associated with neurodegenerative diseases. Common causes of chronic neuroinflammation include: toxic metabolites, autoimmunity, aging, microbes, viruses, traumatic brain injury, spinal cord injury, air pollution, and passive smoke.

Viruses, bacteria, and other infectious agents activate the body's defense systems and cause immune cells to protect the designed area from the damage. Some of these foreign pathogens can trigger a strong inflammatory response that can compromise the integrity of the blood-brain barrier and thus change the flow of inflammation in nearby tissue. The location along with the type of infection can determine what type of inflammatory response is activated and whether specific cytokines or immune cells will act.

Glial cells. Microglia are recognized as the innate immune cells of the central nervous system. Microglia actively survey their environment and change their cell morphology significantly in response to neural injury. Acute inflammation in the brain is typically characterized by rapid activation of microglia. During this period, there is no peripheral immune response. Over time, however, chronic inflammation causes the degradation of tissue and of the blood-brain barrier. During this time, microglia generate reactive oxygen species and release signals to recruit peripheral immune cells for an inflammatory response. Glial cells also play a vital role as house keepers, supplying nutrients, pruning & strengthening synapses, removal of toxic waste and even self-sacrifice in order to protect neurons from foreign invaders.

Astrocytes are glial cells that are the most abundant cells in the brain. They are involved in maintenance and support of neurons and compose a significant component of the blood-brain barrier. After insult to the brain, such as traumatic brain injury, astrocytes may become activated in response to signals released by injured neurons or activated microglia. Once activated, astrocytes may release various growth factors and undergo morphological changes. For example, after injury, astrocytes form the glial scar composed of a proteoglycan matrix that hinders axonal regeneration. However, more recent studies revealed that glia scar is not detrimental, but is in fact beneficial for axonal regeneration.

Cytokines. Cytokines are a class of proteins that regulates inflammation, cell signaling, and various cell processes such as growth and survival. Chemokines are a subset of cytokines that regulate cell migration, such as attracting immune cells to a site of infection or injury. Various cell types in the brain may produce cytokines and chemokines such as microglia, astrocytes, endothelial cells, and other glial cells. Physiologically, chemokines and cytokines function as neuromodulators that regulate inflammation and development. In the healthy brain, cells secrete cytokines to produce a local inflammatory environment to recruit microglia and clear the infection or injury. However, in neuroinflammation, cells may have sustained release of cytokines and chemokines which may compromise the blood-brain barrier. Peripheral immune cells are called to the site of injury via these cytokines and may now migrate across the compromised blood brain barrier into the brain. Common cytokines produced in response to brain injury include: interleukin-6 (IL-6), which is produced during astrogliosis, and interleukin-1 beta (IL-1β) and tumor necrosis factor alpha (TNF-α), which can induce neuronal cytotoxicity. Although the pro-inflammatory cytokines may cause cell death and secondary tissue damage, they are necessary to repair the damaged tissue. For example, TNF-α causes neurotoxicity at early stages of neuroinflammation, but contributes to tissue growth at later stages of inflammation.

Peripheral immune response. The blood-brain barrier is a structure composed of endothelial cells and astrocytes that forms a barrier between the brain and circulating blood. Physiologically, this enables the brain to be protected from potentially toxic molecules and cells in the blood. Astrocytes form tight junctions, and therefore may strictly regulate what may pass the blood-brain barrier and enter the interstitial space. After injury and sustained release of inflammatory factors such as chemokines, the blood-brain barrier may be compromised, becoming permeable to circulating blood components and peripheral immune cells. Cells involved in the innate and adaptive immune responses, such as macrophages, T cells, and B cells, may then enter into the brain. This exacerbates the inflammatory environment of the brain and contributes to chronic neuroinflammation and neurodegeneration.

Traumatic brain injury. Traumatic brain injury (TBI) is brain trauma caused by significant force to the head. Following TBI, there are both reparative and degenerative mechanisms that lead to an inflammatory environment. Within minutes of injury, pro-inflammatory cytokines are released. The pro-inflammatory cytokine Il-1β is one such cytokine that exacerbates the tissue damage caused by TBI. TBI may cause significant damage to vital components to the brain, including the blood-brain barrier. Il-1β causes DNA fragmentation and apoptosis, and together with TNF-α may cause damage to the blood-brain-barrier and infiltration of leukocytes. Increased density of activated immune cells have been found in the human brain after concussion.

As the most abundant immune cells in the brain, Microglia are important to the brain's defense against injury. The major caveat of these cells comes from the fact that their ability to promote recovery mechanism with anti-inflammatory factors, is inhibited by their secondary ability to make a large amount of pro-inflammatory cytokines. This can result in sustained brain damage as anti-inflammatory factors decrease in amount when more pro-inflammatory cytokines are produced in excess by microglia. The cytokines produced by microglia, astrocytes, and other immune cells, activate glial cells further increasing the number of pro-inflammatory factors that further prevent neurological systems from recovering. The dual nature of microglia is one example of why neuroinflammation can be helpful or hurtful under specific conditions.

Spinal Cord Injury (SCI) can be divided into three separate phases. The primary or acute phase occurs from seconds to minutes after injury, the secondary phase occurs from minutes to weeks after injury, and the chronic phase occurs from months to years following injury. A primary SCI is caused by spinal cord compression or transection, leading to glutamate excitotoxicity, sodium and calcium ion imbalances, and free radical damage. Neurodegeneration via apoptosis and demyelination of neuronal cells causes inflammation at the injury site. This leads to a secondary SCI, whose symptoms include edema, cavitation of spinal parenchyma, reactive gliosis, and potentially permanent loss of function.

During the SCI induced inflammatory response, several pro-inflammatory cytokines including interleukin 1β (IL-1β), inducible Nitric Oxide Synthase (iNOS), Interferon-γ (IFN-γ), IL-6, IL-23, and tumor necrosis factor α (TNFα) are secreted, activating local microglia and attracting various immune cells such as naive bone-marrow derived macrophages. These activated microglia and macrophages play a role in the parthenogenesis of SCI.

Upon infiltration of the injury site's epicenter, macrophages will undergo phenotype switching from an M2 phenotype to an M1-like phenotype. The M2 phenotype is associated with anti-inflammatory factors such as IL-10, IL-4, and IL-13 and contributes to wound healing and tissue repair. However, the M1-like phenotype is associated with pro-inflammatory cytokines and reactive oxygen species that contribute to increased damage and inflammation. Factors such as myelin debris, which is formed by the injury at the damage site, has been shown to induce the phenotype shift from M2 to M1. A decreased population of M2 macrophages and an increased population of M1 macrophages is associated with chronic inflammation. Short term inflammation is important in clearing cell debris from the site of injury, but it is this chronic, long-term inflammation that will lead to further cell death and damage radiating from the site of injury.

Aging. Aging is often associated with cognitive impairment and increased propensity for developing neurodegenerative diseases, such as Alzheimer's disease. Elevated inflammatory markers seemed to accelerate the brain aging process. In the aged brain alone, without any evident disease, there are chronically increased levels of pro-inflammatory cytokines and reduced levels of anti-inflammatory cytokines. The homeostatic imbalance between anti-inflammatory and pro-inflammatory cytokines in aging is one factor that increases the risk for neurodegenerative disease. Additionally, there is an increased number of activated microglia in aged brains, which have increased expression of major histocompatibility complex II (MHC II), ionized calcium binding adaptor-1 (IBA1), CD86, ED1 macrophage antigen, CD4, and leukocyte common antigen. These activated microglia decrease the ability for neurons to undergo long term potentiation (LTP) in the hippocampus and thereby reduce the ability to form memories. LTP can happen in most parts of brain, typically by pulses and activation of AMPA receptors. Neural progenitor cells (NPC's) and new brain cells are always being made, hippocampus is one hot spot for the neurogenesis. Even though we make new neurons every day, they rarely reach maturity unless properly stimulated. Then, the new neurons slowly move towards dengate gyrus in roughly 18 days to be cemented in.

Monoamine oxidase A, also known as MAO-A, is an enzyme that in humans is encoded by the MAO-A gene. This gene is one of two neighboring gene family members that encode mitochondrial enzymes which catalyze the oxidative deamination of amines, such as dopamine, norepinephrine, and serotonin. A mutation of this gene results in Brunner syndrome. This gene has also been associated with a variety of other psychiatric disorders, including antisocial behavior. Alternatively spliced transcript variants encoding multiple isoforms have been observed. The promoter of MAO-A contains conserved binding sites for Sp1, GATA2, and TBP. This gene is adjacent to a related gene (MAO-B) on the opposite strand of the X chromosome.

In humans, there is a 30-base repeat sequence repeated several different numbers of times in the promoter region of MAO-A. There are 2R (two repeats), 3R, 3.5R, 4R, and 5R variants of the repeat sequence, with the 3R and 4R variants most common in all populations. The 3.5R and 4R variants have been found to be more highly active than 3R or 5R, in a study which did not examine the 2R variant.

The gene encodes a monomeric protein which shares a 70% amino acid sequence identity, as well as conserved chain folds and flavin adenine dinucleotide (FAD)-binding site structures, with MAO-B. However, MAO-A has a monopartite cavity of approximately 550 angstroms, compared to the 290-angstrom bipartite cavity in MAO-B. Nonetheless, both proteins adopt dimeric forms when membrane-bound. The C-terminal domain of MAO-A forms helical tails which are responsible for attaching the protein to the outer mitochondrial membrane (OMM). MAO-A contains loop structures at the entrance of its active site.

MAO-A is a key regulator for normal brain function. It is a flavoenzyme which degrades amine neurotransmitters, such as dopamine, norepinephrine, and serotonin, via oxidative deamination. It is highly expressed in neural and cardiac cells and localizes to the outer mitochondrial membrane. Its expression is regulated by the transcription factors SP1, GATA2, and TBP via the CAMP pathway in response to stress such as ischemia and inflammation. One behavioral study suggested a link between the 2R allele and a higher likelihood of violent behavior in adolescents and young adults while another showed no link at all with either antisocial behavior or antisocial alcoholism.

There is some association between low activity forms of the MAO-A gene and autism. Mutations in the MAO-A gene results in monoamine oxidase deficiency, or Brunner syndrome. Other disorders associated with MAO-A include Alzheimer's disease, aggression, panic disorder, bipolar affective disorder, major depressive disorder, and attention deficit hyperactivity disorder. Effects of parenting on self-regulation in adolescents appear to be moderated by ‘plasticity alleles’, of which the 2R and 3R alleles of MAO-A are two, with the more plasticity alleles males (but not females) carried, the more and less self-regulation they manifested under, respectively, supportive and unsupportive parenting conditions.

MAO-A levels in the brain as measured using positron emission tomography are elevated by an average of 34% in patients with major depressive disorder. Genetic association studies examining the relationship between high-activity MAO-A variants and depression have produced mixed results, with some studies linking the high-activity variants to major depression in females, depressed suicide in males, major depression and sleep disturbance in males and major depressive disorder in both males and females.

Other studies failed to find a significant relationship between high-activity variants of the MAO-A gene and major depressive disorder. In patients with major depressive disorder, those with MAO-A G/T polymorphisms (rs6323) coding for the highest-activity form of the enzyme have a significantly lower magnitude of placebo response than those with other genotypes.

A dysfunctional MAO-A gene has been correlated with increased aggression levels in mice, and has been correlated with heightened levels of aggression in humans. In mice, a dysfunctional MAO-A gene is created through insertional mutagenesis (called ‘Tg8’). Tg8 is a transgenic mouse strain that lacks functional MAO-A enzymatic activity. Mice that lacked a functional MAO-A gene exhibited increased aggression towards intruder mice.

Some types of aggression exhibited by these mice were territorial aggression, predatory aggression, and isolation-induced aggression. The MAO-A deficient mice that exhibited increased isolation-induced aggression reveals that an MAO-A deficiency may also contribute to a disruption in social interactions. There is research in both humans and mice to support that a non-sensing point mutation in the eighth exon of the MAO-A gene is responsible for impulsive aggressiveness due to a complete MAO-A deficiency.

Interleukin 13 (IL-13) is a protein that in humans is encoded by the IL-13 gene. IL-13 was first cloned in 1993 and is located on chromosome 5q31 with a length of 1.4 kb. It has a mass of 13 kDa and folds into 4-alpha helical bundles. The secondary structural features of IL-13 are similar to that of Interleukin 4 (IL-4); however it only has 25% sequence identity to IL-4 and is capable of IL-4 independent signaling. IL-13 is a cytokine secreted by T helper type 2 (Th2) cells, CD4 cells, natural killer T cell, mast cells, basophils, eosinophils and nuocytes. Interleukin-13 is a central regulator in IgE synthesis, goblet cell hyperplasia, mucus hyper-secretion, airway hyper-responsiveness, fibrosis and chitinase up-regulation. It is a mediator of allergic inflammation and different diseases including asthma.

IL-13 has effects on immune cells that are similar to those of the closely related cytokine IL-4. However, IL-13 is suspected to be the central mediator of the physiologic changes induced by allergic inflammation in many tissues.

Although IL-13 is associated primarily with the induction of airway disease, it also has anti-inflammatory properties. IL-13 induces a class of protein-degrading enzymes, known as matrix metal-loproteinases (MMPs), in the airways. These enzymes are required to induce aggression of parenchymal inflammatory cells into the airway lumen, where they are then cleared. Among other factors, IL-13 induces these MMPs as part of a mechanism that protects against excessive allergic inflammation that predisposes to asphyxiation.

IL-13 is known to induce changes in hematopoietic cells, but these effects are probably less important than that of IL-4. Furthermore, IL-13 can induce immunoglobulin E (IgE) secretion from activated human B cells. Deletion of IL-13 from mice does not markedly affect either Th2 cell development or antigen-specific IgE responses induced by potent allergens. In comparison, deletion of IL-4 deactivates these responses. Thus, rather than a lymphoid cytokine, IL-13 acts more prominently as a molecular bridge linking allergic inflammatory cell to the non-immune cells in contact with them, thereby altering physiological function.

The signaling of IL-13 begins through a shared multi-subunit receptor with IL-4. This receptor is a heterodimer receptor complex consisting of alpha IL-4 receptor (IL-4Ra) and alpha Interleukin-13 receptor (IL-13R1). The high affinity of IL-13 to the IL-13R1 leads to their bond formation which further increase the probability of a heterodimer formation to IL-4R1 and the production of the type 2 IL-4 receptor. Heterodimerization activates both the STAT6 and the IRS. STAT6 signaling is important in initiation of the allergic response. Most of the biological effects of IL-13, like those of IL-4, are linked to a single transcription factor, signal transducer and activator of transcription 6 (STAT6). Interleukin-13 and its associated receptors with a subunit of the IL-4 receptor (IL-4Ra) allows for the downstream activation of STAT6. The JAK Janus kinase proteins on the cytoplasmic end of the receptors allows for the phosphorylation of STAT6, which then forms an activated homodimer and are transported to the nucleus. Once, in the nucleus, STAT6 heterodimer molecule regulates gene expression of cell types critical to the balance between host immune defense and allergic inflammatory responses such as the development of Th2. This can be resulted from an allergic reaction brought about when facing an Ala gene. IL-13 also binds to another receptor known as IL-13Rα2. IL-13Rα2 (which is labeled as a decoy receptor) is derived from Th2 cells and is a pleotropic immune regulatory cytokine. IL-13 has greater affinity (50-times) to IL-13Rα2 than to IL-13Ra1. The IL-13Rα2 subunit binds only to IL-13 and it exists in both membrane-bound and soluble forms in mice. A soluble form of IL-13Rα2 has not been detected in human subjects. Studies of IL-13 transgenic mice lungs with IL-13Rα2 null loci indicated that IL-13Rα2 deficiency significantly augmented IL-13 or ovalbumin-induced pulmonary inflammation and remodeling. Most normal cells, such as immune cells or endothelial cells, express very low or undetectable levels of IL-13 receptors. Research has shown that cell-surface expression of IL-13Rα2 on human asthmatic airway fibroblasts was reduced compared with expression on normal control airway fibroblasts. This supported the hypothesis that IL-13Rα2 is a negative regulator of IL-13-induced response and illustrated significantly reduced production of TGF-β1 and deposition of collagen in the lungs of mice.

Interleukin-13 has a critical role in the Goblet cell metaplasia. Goblet cells are filled with mucin (MUC). MUC5AC Mucin 5AC is a gel-like mucin product of goblet cells. Interleukin-13 induces goblet cell differentiation and allows for the production of MUC5AC in tracheal epithelium. 15-Lipoxygenase-1 (15LO1) which is an enzyme in the fatty acid metabolism and its metabolite, 15-HETE, are highly expressed in asthma (which lead to the overexpression of MUC5AC) and are induced by IL-13 in human airway epithelial cells. With the increasing number of goblet cells, there is the production of excessive mucus within the bronchi. The functional consequences of the changes in MUC storation and secretion contributes to the pathophysiologic mechanisms for various clinical abnormalities in asthmatic patients including sputum production, airway narrowing, exacerbation and accelerated loss in lung function.

Additionally, IL-13 has been shown to induce a potent fibrogenic program during the course of diverse diseases marked by elevated Type 2 cytokines such as chronic schistosomiasis and atopic dermatitis among others. It has been suggested that this fibrogenic program is critically dependent on direct IL-13 signaling through IL-4Ra on PDGFRβ+ fibroblasts.

Signal transducer and activator of transcription 3 (STAT3) is a transcription factor which in humans is encoded by the STAT3 gene. It is a member of the STAT protein family.

STAT3 is a member of the STAT protein family. In response to cytokines and growth factors, STAT3 is phosphorylated by receptor-associated Janus kinases (JAK), forms homo- or heterodimers, and translocates to the cell nucleus where it acts as a transcription activator. Specifically, STAT3 becomes activated after phosphorylation of tyrosine 705 in response to such ligands as interferons, epidermal growth factor (EGF), Interleukin (IL-)5 and IL-6. Additionally, activation of STAT3 may occur via phosphorylation of serine 727 by Mitogen-activated protein kinases (MAPK)[6] and through c-src non-receptor tyrosine kinase. STAT3 mediates the expression of a variety of genes in response to cell stimuli, and thus plays a key role in many cellular processes such as cell growth and apoptosis.

STAT3-deficient mouse embryos cannot develop beyond embryonic day 7, when gastrulation begins. It appears that at these early stages of development, STAT3 activation is required for self-renewal of embryonic stem cells (ESCs). Indeed, LIF, which is supplied to murine ESC cultures to maintain their undifferentiated state, can be omitted if STAT3 is activated through some other means.

STAT3 is essential for the differentiation of the TH17 helper T cells, which have been implicated in a variety of autoimmune diseases. During viral infection, mice lacking STAT3 in T-cells display impairment in the ability to generate T-follicular helper (Tfh) cells and fail to maintain antibody based immunity.

STAT3 caused upregulation in E-selectin, a factor in metastasis of cancers. Hyperactivation of STAT3 occurs in COVID-19 infection and other viral infections.

White matter refers to areas of the central nervous system (CNS) that are mainly made up of myelinated axons, also called tracts. Long thought to be passive tissue, white matter affects learning and brain functions, modulating the distribution of action potentials, acting as a relay and coordinating communication between different brain regions.

White matter is named for its relatively light appearance resulting from the lipid content of myelin. However, the tissue of the freshly cut brain appears pinkish-white to the naked eye because myelin is composed largely of lipid tissue veined with capillaries. Its white color in prepared specimens is due to its usual preservation in formaldehyde.

White matter is composed of bundles, which connect various grey matter areas (the locations of nerve cell bodies) of the brain to each other, and carry nerve impulses between neurons. Myelin acts as an insulator, which allows electrical signals to jump, rather than coursing through the axon, increasing the speed of transmission of all nerve signals.

The total number of long range fibers within a cerebral hemisphere is 2% of the total number of cortico-cortical fibers (across cortical areas) and is roughly the same number as those that communicate between the two hemispheres in the brain's largest white tissue structure, the corpus callosum. As a rough rule, the number of fibres of a certain range of lengths is inversely proportional to their length. White matter in non-elderly adults is 1.7-3.6% blood.

A demyelinating disease is any disease of the nervous system in which the myelin sheath of neurons is damaged. This damage impairs the conduction of signals in the affected nerves. In turn, the reduction in conduction ability causes deficiency in sensation, movement, cognition, or other functions depending on which nerves are involved.

Demyelinating diseases can be caused by genetics, infectious agents, autoimmune reactions, and other unknown factors. Proposed causes for demyelination include genetics and environmental factors such as being triggered by a viral infection or chemical exposure. Organophosphate poisoning by commercial insecticides such as sheep dip, weed killers, and flea treatment preparations for pets, can also result in nerve demyelination. Chronic neuroleptic exposure may cause demyelination. Vitamin B12 deficiency may also result in dysmyelination.

Demyelinating diseases are traditionally classified in two kinds: demyelinating myelinoclastic diseases and demyelinating leukodystrophic diseases. In the first group, a normal and healthy myelin is destroyed by a toxic, chemical, or autoimmune substance. In the second group, myelin is abnormal and degenerates. The second group was denominated dysmyelinating diseases.

In the most well known example of demyelinating disease, multiple sclerosis, evidence has shown that the body's own immune system is at least partially responsible. Acquired immune system cells called T-cells are known to be present at the site of lesions. Other immune-system cells called macrophages (and possibly mast cells) also contribute to the damage.

Peroxisome proliferator-activated receptor gamma coactivator 1-alpha (PGC-1α) is a protein that in humans is encoded by the PPARGC1A gene. PPARGC1A is also known as human accelerated region 20 (HAR20). It may, therefore, have played a key role in differentiating humans from apes.

PGC-1α is the master regulator of mitochondrial biogenesis. PGC-1α is also the primary regulator of liver gluconeogenesis, inducing increased gene expression for gluconeogenesis.

PGC-1α is a transcriptional coactivator that regulates the genes involved in energy metabolism. It is the master regulator of mitochondrial biogenesis. This protein interacts with the nuclear receptor PPAR-γ, which permits the interaction of this protein with multiple transcription factors. This protein can interact with, and regulate the activity of, cAMP response element-binding protein (CREB) and nuclear respiratory factors (NRFs). PGC-1α provides a direct link between external physiological stimuli and the regulation of mitochondrial biogenesis, and is a major factor causing slow-twitch rather than fast-twitch muscle fiber types.

Endurance exercise has been shown to activate the PGC-1α gene in human skeletal muscle. Exercise-induced PGC-1α in skeletal muscle increases autophagy and unfolded protein response. PGC-1α protein may be also involved in controlling blood pressure, regulating cellular cholesterol homoeostasis, and the development of obesity. PGC-1α is thought to be a master integrator of external signals. It is known to be activated by a host of factors, including: Reactive oxygen species (ROS) and reactive nitrogen species (RNS), both formed endogenously in the cell as by-products of metabolism but upregulated during times of cellular stress. Fasting can also increase gluconeogenic gene expression, including hepatic PGC-1α.

It is strongly induced by cold exposure, linking this environmental stimulus to adaptive thermogenesis. It is induced by endurance exercise and recent research has shown that PGC-1α determines lactate metabolism, thus preventing high lactate levels in endurance athletes and making 1αacetate as an energy source more efficient. cAMP response element-binding (CREB) proteins, activated by an increase in cAMP following external cellular signals. Protein kinase B/Akt is thought to downregulate PGC-1α, but upregulate its downstream effectors, NRF-1 and NRF-2. Akt itself is activated by PIP3, often upregulated by PI3K after G-protein signals. The Akt family is also known to activate pro-survival signals as well as metabolic activation. SIRT1 binds and activates PGC-1α through deacetylation inducing gluconeogenesis without affecting mitochondrial biogenesis. Increases generation of “brown fat”.

PGC-1α has been shown to exert positive feedback circuits on some of its upstream regulators. PGC-1α increases Akt (PKB) and Phospho-Akt (Ser 473 and Thr 308) levels in muscle.

PGC-1α leads to calcineurin activation. Akt and calcineurin are both activators of NF kappa B (p65). Through their activation PGC-1α seems to activate NF kappa B. Increased activity of NF kappa B in muscle has recently been demonstrated following induction of PGC-1α. The finding seems to be controversial. Other groups found that PGC-1s inhibit NF kappa B activity. The effect was demonstrated for PGC-1 alpha and beta. PGC-1α has also been shown to drive NAD biosynthesis to play a large role in renal protection in Acute Kidney Injury.

Nuclear respiratory factor 1, also known as NRF-1, encodes a protein that homodimerizes and functions as a transcription factor which activates the expression of some key metabolic genes regulating cellular growth and nuclear genes required for respiration, heme biosynthesis, and mitochondrial DNA transcription and replication. The protein has also been associated with the regulation of neurite outgrowth. Alternate transcriptional splice variants, which encode the same protein, have been characterized. Additional variants encoding different protein isoforms have been described but they have not been fully characterized. Confusion has occurred in bibliographic databases due to the shared symbol of NRF-1 for this gene and for nuclear factor (erythroid-derived 2)-like 1 which has an official symbol of NFE2L1.

Nuclear factor erythroid 2-related factor 2 (NRF-2), also known as nuclear factor erythroid-derived 2-like 2, is a transcription factor (master antioxidant transcription factor and Redox-sensitive like NFK) that in humans is encoded by the NFE2L2 gene. NRF-2 is a basic leucine zipper (bZIP) protein that may regulate the expression of antioxidant proteins that protect against oxidative damage triggered by injury and inflammation, according to preliminary research. In vitro, NRF-2 binds to antioxidant response elements (AREs) in the promoter regions of genes encoding cytoprotective proteins. NRF-2 induces the expression of heme oxygenase 1 in vitro leading to an increase in phase II enzymes. NRF-2 also inhibits the NLRP3 inflammasome.

NRF-2 appears to participate in a complex regulatory network and performs a pleiotropic role in the regulation of metabolism, inflammation, autophagy, proteostasis, mitochondrial physiology, and immune responses. Several drugs that stimulate the NFE2L2 pathway are being studied for treatment of diseases that are caused by oxidative stress.

A mechanism for hormetic dose responses is proposed in which NRF-2 may serve as an hormetic mediator that mediates a vast spectrum of chemopreventive processes. 5′ AMP-activated protein kinase or AMPK or 5′ adenosine monophosphate-activated protein kinase is an enzyme (EC 2.7.11.31) that plays a role in cellular energy homeostasis, largely to activate glucose and fatty acid uptake and oxidation when cellular energy is low. It belongs to a highly conserved eukaryotic protein family and its orthologues are SNF1 in yeast, and SnRK1 in plants. It consists of three proteins (subunits) that together make a functional enzyme, conserved from yeast to humans. It is expressed in a number of tissues, including the liver, brain, and skeletal muscle. In response to binding AMP and ADP, the net effect of AMPK activation is stimulation of hepatic fatty acid oxidation, ketogenesis, stimulation of skeletal muscle fatty acid oxidation and glucose uptake, inhibition of cholesterol synthesis, lipogenesis, and triglyceride synthesis, inhibition of adipocyte lipogenesis, inhibition of adipocyte lipolysis, and modulation of insulin secretion by pancreatic β-cells.

It should not be confused with cyclic AMP-activated protein kinase (protein kinase A). Superoxide dismutase 2, mitochondrial (SOD2), also known as manganese-dependent superoxide dismutase (MnSOD), is an enzyme which in humans is encoded by the SOD2 gene on chromosome 6. A related pseudogene has been identified on chromosome 1. Alternative splicing of this gene results in multiple transcript variants. This gene is a member of the iron/manganese superoxide dismutase family. It encodes a mitochondrial protein that forms a homotetramer and binds one manganese ion per subunit. This protein binds to the superoxide byproducts of oxidative phosphorylation and converts them to hydrogen peroxide and diatomic oxygen. Mutations in this gene have been associated with idiopathic cardiomyopathy (IDC), premature aging, sporadic motor neuron disease, and cancer.

Catalase is a common enzyme found in nearly all living organisms exposed to oxygen (such as bacteria, plants, and animals) which catalyzes the decomposition of hydrogen peroxide to water and oxygen. It is a very important enzyme in protecting the cell from oxidative damage by reactive oxygen species (ROS). Catalase has one of the highest turnover numbers of all enzymes; one catalase molecule can convert millions of hydrogen peroxide molecules to water and oxygen each second.

Catalase is a tetramer of four polypeptide chains, each over 500 amino acids long. It contains four iron-containing heme groups that allow the enzyme to react with hydrogen peroxide. The optimum pH for human catalase is approximately 7, and has a fairly broad maximum: the rate of reaction does not change appreciably between pH 6.8 and 7.5. The pH optimum for other catalases varies between 4 and 11 depending on the species. The optimum temperature also varies by species.

Nuclear respiratory factor 1, also known as NRF-1, encodes a protein that homodimerizes and functions as a transcription factor which activates the expression of some key metabolic genes regulating cellular growth and nuclear genes required for respiration, heme biosynthesis, and mitochondrial DNA transcription and replication. The protein has also been associated with the regulation of neurite outgrowth. Alternate transcriptional splice variants, which encode the same protein, have been characterized. Additional variants encoding different protein isoforms have been described but they have not been fully characterized. Confusion has occurred in bibliographic databases due to the shared symbol of NRF-1 for this gene and for nuclear factor (erythroid-derived 2)-like 1 which has an official symbol of NFE2L1.

NRF-1 functions as a transcription factor that activates the expression of some key metabolic genes regulating cellular growth and nuclear genes required for mitochondrial respiration, and mitochondrial DNA transcription and replication. NRF-1, together with NRF-2, mediates the biogenomic coordination between nuclear and mitochondrial genomes by directly regulating the expression of several nuclear-encoded ETC proteins, and indirectly regulating the three mitochondrial-encoded COX subunit genes by activating mtTFA, mtTFB1, and mtTFB2.

NRF-1 is associated with the regulation of neurite outgrowth. Alternate transcriptional splice variants, which encode the same protein, have been characterized. Additional variants encoding different protein isoforms have been described but they have not been fully characterized. Cyclin D1-dependent kinase, through phosphorylating NRF-1 at S47, coordinates nuclear DNA synthesis and mitochondrial function.

NF-κB (or NF-kappaB, “nuclear factor kappa-light-chain-enhancer of activated B cells”) is a protein complex that controls transcription of DNA, cytokine production and cell survival. NF-κB is found in almost all animal cell types and is involved in cellular responses to stimuli such as stress, cytokines, free radicals, heavy metals, ultraviolet irradiation, oxidized LDL, and bacterial or viral antigens. NF-κB plays a key role in regulating the immune response to infection. Incorrect regulation of NF-κB has been linked to cancer, inflammatory and autoimmune diseases, septic shock, viral infection, and improper immune development. NF-κB has also been implicated in processes of synaptic plasticity and memory.

Mechanism of NF-κB action. The classic “canonical” NF-κB complex is a heterodimer of p50 and RelA, as shown. While in an inactivated state, NF-κB is located in the cytosol complexed with the inhibitory protein IκBα. Through the intermediacy of integral membrane receptors, a variety of extracellular signals can activate the enzyme IκB kinase (IKK). IKK, in turn, phosphorylates the IκBα protein, which results in ubiquitination, dissociation of IκBα from NF-κB, and eventual degradation of IκBα by the proteasome. The activated NF-κB is then translocated into the nucleus where it binds to specific sequences of DNA called response elements (RE). The DNA/NF-κB complex then recruits other proteins such as coactivators and RNA polymerase, which transcribe downstream DNA into mRNA. In turn, mRNA is translated into protein, resulting in a change of cell function.

### SUMMARY OF THE INVENTION

One aspect of the invention is a method of treating chronic or acute conditions associated with specific or non-specific neuroinflammation in a human consisting of inhibiting the MAO-A isoform enzymatic pathway by administering an oral dosage form comprising a therapeutic dose of first and second particulate metaxalones in one or more suitable forms selected from salts, co-crystals, solvates, free form, enantiomers, and polymorphs thereof, and, one or more pharmaceutically suitable excipients, wherein the dose preferably has a stable dissolution profile over time.

Another aspect of the invention is a method of inhibiting the MAO-A enzymatic pathway consisting of administering an oral dosage form comprising a therapeutic dose of first and second particulate metaxalones in one or more suitable forms selected from salts, co-crystals, solvates, free form, enantiomers, and polymorphs thereof, and, one or more pharmaceutically suitable excipients, wherein the dosage form preferably has a stable dissolution profile over time.

Another aspect of the invention is a method of treating chronic or acute conditions associated with specific or non-specific neuroinflammation in a human consisting of administering an oral dosage form comprising a therapeutic dose of first and second particulate metaxalones in one or more suitable forms selected from salts, co-crystals, solvates, free form, enantiomers, and polymorphs thereof, and, one or more pharmaceutically suitable excipients, wherein the dosage form preferably has a stable dissolution profile over time.

A method of restoring or increasing IL-13 and inhibiting STAT3 consisting of administering an oral dosage form comprising a therapeutic dose of first and second particulate metaxalones in one or more suitable forms selected from salts, co-crystals, solvates, free form, enantiomers, and polymorphs thereof, and, one or more pharmaceutically suitable excipients, wherein the dosage form preferably has a stable dissolution profile over time.

In another exemplary embodiment, the treatment method further includes increasing white matter growth and development in the central nervous system by increasing and/or attenuating the natural pathways responsible for proper white matter cellular growth and repair.

In another exemplary embodiment, the treatment method further includes treating demyelination disorders or diseases.

Another aspect of the invention is a method of increasing or restoring PGC-1α consisting of administering an oral dosage form comprising a therapeutic dose of first and second particulate metaxalones in one or more suitable forms selected from salts, co-crystals, solvates, free form, enantiomers, and polymorphs thereof, and, one or more pharmaceutically suitable excipients. Fibromyalgia, sciatica, neuropathic pain and stroke/TBI have decreased PGC-1α levels which prevents the restoration, new mitochondria growth (mitochondrial biogenesis), and repair mechanisms for damaged/mutated mtDNA. After a stroke for instance, PGC-1α sky rockets for first 24 hours after incident, in order to try and restore neural function, restore autophagy, antioxidant pathways and repair. Although after 24 hours the highly important activation of PGC-1α drops significantly and significantly lowers repair systems of the brain.

In another exemplary embodiment, the treatment method further includes treating one or more disorders or diseases selected from sciatica, fibromyalgia, neuropathic pain, ischemic stroke and traumatic brain injury.

In another exemplary embodiment, neuropathic pain is selected from mechanical allodynia, thermal hyperalgesia, diabetic neuropathy, chemotherapy neuropathy and post-operative pain.

In another exemplary embodiment, the treatment method further includes restoring mitochondrial biogenesis.

In another exemplary embodiment, the treatment method further includes stimulating NRF-1 and NRF-2.

In another exemplary embodiment, the treatment method further includes preventing chronification of neuropathic pain due to haploinsufficiency.

In another exemplary embodiment, the treatment method further includes stimulating AMPK.

In another exemplary embodiment, the treatment method further includes bolstering gene expression and protein syntheses of MnSOD and catalase.

In another exemplary embodiment, the treatment method further includes treating cardiovascular disorder or disease and treating vascular leakage caused by permeability.

In another exemplary embodiment, the treatment method further includes treating pain caused by nerve injury.

In another exemplary embodiment, the treatment method further includes preventing pain chronification caused by burn injury.

Another aspect of the invention is a method of treating chronic venous insufficiency and increasing AMPK and PGC-1α and restoring MnSOD and catalase consisting of administering an oral dosage form comprising a therapeutic dose of first and second particulate metaxalones in one or more suitable forms selected from salts, co-crystals, solvates, free form, enantiomers, and polymorphs thereof, and, one or more pharmaceutically suitable excipients, wherein the dosage form preferably has a stable dissolution profile over time.

In another exemplary embodiment, the treatment method further includes inhibiting NF-κB.

In another exemplary embodiment, the treatment method further includes revitalizing muscle strength.

In another exemplary embodiment, the treatment method further includes initiating regeneration of muscular tissue.

In another exemplary embodiment, the treatment method further includes promoting an antifibrotic state.

In another exemplary embodiment, the treatment method further includes preventing carpal tunnel fibrosis.

In an exemplary embodiment, the excipients are selected from alginic acid, propylene glycol alginate and combinations thereof.

In another exemplary embodiment, the therapeutic dose in the range of 600-700 mg of metaxalone.

In another exemplary embodiment, the therapeutic dose is ˜640 mg of metaxalone.

In another exemplary embodiment, the oral dosage form contains 40-80% wt of a first particulate metaxalone and 20-60% wt of a second particulate metaxalone, wherein ≥90% wt of the first metaxalone has a particle size of ˜7-86μ, and, wherein ≥35% wt of the second metaxalone is has a particle size in the range of ˜98 to 516μ.

Still other embodiments relate to tablets capable of achieving the stable dissolution profile depicted in the figures hereto. As described herein, this stable dissolution profile is achieved by properly balancing the hardness of the tablet with the particle size of the metaxalone. Thus, in one embodiment the tablets of the current invention comprise means for maintaining a stable dissolution profile, wherein the means corresponds to a balance of hardness and API particle size capable of maintaining the dissolution profile when tested according to the methods described in in the examples hereto or USP [Metaxalone Tablets]. In a particularly preferred embodiment, the percentage of metaxalone released after 6 months differs by no more than 5%, 3%, or 2%, on an absolute basis, from the percentage of metaxalone released after 0 months or 3 months, when stored at 40±2° C. and 75%±5% relative humidity, preferably at 30 and 90 minute time points.

The hardness values for the tablet will commonly range from 6 to 35 kp, from 6 to 25 kp, or from 6 to 17 kp, tested generally, as with all testing of the current invention, according to the version of USP in effect as of the original filing date of this utility application, specifically in accordance with Tablet Breaking Force <1217>.

In another embodiment, the percentage of metaxalone released after 3 months differs by no more than 2%, on an absolute basis, from the percentage of metaxalone released after 0 months, when stored at 40±2° C. and 75%±5% relative humidity, preferably at 30 and 90 minute timepoints when tested according to USP [Metaxalone Tablets].

In still another embodiment, the percentage of metaxalone released after 6 months differs by no more than 3% or 2%, on an absolute basis, from the percentage of metaxalone released after 3 months, when stored at 40±2° C. and 75%±5% relative humidity, preferably at 30 and 60 minute timepoints when tested according to USP [Metaxalone Tablets].

The “absolute basis” of different percentages is determined by subtracting the percent released at one time point from the percent released at another time point. It will be understood that the dissolution testing is performed in accordance with ICH guidelines governing product release, based on a suitable number of tablets, as described in ICH Q6A specifications: test procedures and acceptance criteria for new drug substances and new drug products: chemical substances.

In another exemplary embodiment, the chronic or acute, specific or non-specific neuroinflammatory condition is diabetic neuropathy.

In another exemplary embodiment, the chronic or acute, specific or non-specific, neuroinflammatory condition is fibromyalgia or back pain.

In another exemplary embodiment, the treatment method furthering includes substantially inducing or restoring homeostasis.

In another exemplary embodiment, the treatment method further includes substantially restoring oxidative homeostasis.

In another exemplary embodiment, the treatment method further includes substantially restoring basal phenotype from a neuroinflammatory state.

In another exemplary embodiment, the treatment method further includes treating chronic pain induced depression.

In another exemplary embodiment, the treatment method further includes treating restless leg syndrome.

In another exemplary embodiment, the treatment method further includes improving neuroplasticity.

In another exemplary embodiment, the treatment method further includes treating pain induced depression.

In another exemplary embodiment, the treatment method further includes treating anxiety and mood disorder.

In another exemplary embodiment, the treatment method further includes treating peripheral neuropathy.

In another exemplary embodiment, the treatment method further includes treating herpetic neuralgia.

In another exemplary embodiment, the micronized and non-micronized particulate metaxalones are a crystalline racemic mixture.

In another exemplary embodiment, the oral dosage form induces a pharmacokinetic profile in adult males, under fasted conditions, of Tₘₐₓ˜1.5-12 hr, mean Cₘₐₓ˜2 mcg/ml, AUC˜16 mcg·h/ml, and T_{1/2}˜5 hr, and wherein said AUC_{0→∞} and mean Cₘₐₓ in adult females, under fasted conditions, is ˜40% greater as compared to adult males.

In another exemplary embodiment, the oral dosage form is administered without food.

In another exemplary embodiment, the oral dosage form is co-administered with food.

In another exemplary embodiment, the food effect of high fat meal is ˜23% increase in Cₘₐₓ, no change in AUC_{0→∞}, and Tₘₐₓ˜3.5-24 hr.

In another exemplary embodiment, for fasted adult males, Tₘₐₓ˜3.0 hr.

In another exemplary embodiment, Tₘₐₓ˜8 hr.

In another exemplary embodiment, the oral dosage form is administered 3 or 4 times daily.

As demonstrated by the examples herein, metaxalone is shown to significantly restore and increase IL-13. IL-13 is an inhibitor of STAT3. Inhibiting STAT3 causes an increase in white matter growth and development in the CNS. For patients with demyelination disorders or diseases, application of metaxalone use may be of use for increasing and restoring white matter in the brain. Further, the examples herein demonstrate that metaxalone significantly increased the mitochondrial biogenesis gene, peroxisome proliferator-activated receptor gamma coactivator-1 alpha (PGC-1α).

Specifically PGC-1 is significantly altered in patients with Sciatica (decreased PGC-1α), fibromyalgia (decreased PGC-1α), neuropathic pain (decreased PGC-1α), mechanical allodynia, thermal hyperalgesia, diabetic neuropathy, chemotherapy neuropathy, post-operative pain, ischemic stroke and traumatic brain injury.

PGC-1α and its downstream signaling intermediates are important in normal Mt regulation in diabetic subjects. PGC-1α insufficiency exacerbates glucose intolerance in diabetic patients. Faulty mitochondria leads to inability to process sugars and lipids, as well as inhibiting the use and production of the energy molecule ATP. After 24 hours from induction of stroke the levels of PGC-1α drastically decline, inhibiting restoration of neural pathways and cellular repair mechanisms. Due to the spasticity observed in patients who have had a stroke, the use of typical muscle relaxers is not advised due to increased risk of fall and cognitive decline due to their MOA being related to CNS depression either through A2 agonism or affecting the Gaba a, b, and c receptors (benzodiazepine receptor-(allosteric modulation), barbiturate site (direct binding) and baclofen site (Gaba b)). Thus, metaxalone advantageously supports recovery with less risk of drug interactions because metaxalone that does not function through the Gaba/glutamate channels.

Restoration of mitochondrial biogenesis is crucial in both early and late stages of Traumatic Brain Injury (TBI). Further, restoration of IL-13 increases the white matter synthesis which is vital for neural connections to properly function and send signals.

Metaxalones restores PGC-1α and mitochondrial biogenesis, which leads to treating neuropathic pain. Metaxalone also stimulates NRF-1/NRF-2 which increases mitochondrial health and restores the body's antioxidant status after acute/chronic injury. Metaxalone further prevents the chronification of pain due to PGC-1α haploinsufficiency. Still further, metaxalone stimulates AMPK, which restores mitochondrial biogenesis. Activation of AMPK activation bolsters the gene expression and protein synthesis of MnSOD and catalase. Patients having cardiovascular disease and conditions and patients having a thin blood brain barrier are protected from vascular leakage due to permeability. Metaxalone also treat pain from injured nerves and prevention of pain chronification after burn injury.

By increasing AMPK/PGC-1α in patients with pain due to chronic venous insufficiency MnSOD/catalase is restored (both antioxidant enzymes are vital for vascular integrity to withstand ROS attacks).

Management of inflammation and pain in patients with damage-induced muscle degeneration. PGC-1α inhibits the inflammatory transcription factor NFKB, revitalizes muscle strength, initiates the regeneration of muscular tissue, and promotes an antifibrotic state. Metaxalone also prevents carpal tunnel fibrosis from overuse.

The examples herein demonstrates that muscles with higher levels of PGC-1α are preconditioned for faster resolution of inflammation and necrosis as well as a prevention of fibrosis after repeated injury. Besides its central role in exercise adaptation, the effect of PGC-1α on regeneration contributes to the therapeutic potential of elevation of PGC-1α in different muscle diseases.

### BRIEF DESCRIPTION OF THE DRAWINGS

FIG. 1. Effects of metaxalone on TNF-α (A), IL-6 (B), and IL-13 (C) mRNA expression. Effects of metaxalone on TNF-α (D), IL-6 (E), and IL-13 (F) protein levels. Values are expressed as the means±SD. *, p<0.0001 vs. CTRL; #, p<0.0001 vs. IL-1β.

FIG. 2. Effects of metaxalone on MDA generation (A), NRF-2 mRNA expression (B), and NRF-2 protein levels (C). Values are expressed as the means±SD. *, p<0.0001 vs. CTRL; #, p<0.0001 vs. IL-1β.

FIG. 3. Effects of metaxalone on NF-kB (A), PPARγ (B), and PGC-1α (C) mRNA expression. Effects of metaxalone on pNF-kB (D), PPARγ (E), and PGC-1α (F) protein levels. Values are expressed as the means±SD. *, p<0.0001 vs. CTRL; #, p<0.0001 vs. IL-1.

FIG. 4. Effects of metaxalone on MAO-A mRNA expression (A), MAO-A protein levels (B), MAO-A activity (C). Values are expressed as the means±SD. *, p<0.0001 vs. CTRL; #, p<0.0001 vs. IL1β.

FIG. 5. Effects of metaxalone on cell viability at 24 h (A), 48 h (B), and 72 h (C). Values are expressed as the means±SD. *, p<0.0001 vs. CTRL.

### DETAILED DESCRIPTION OF THE INVENTION

The structure of metaxalone is shown below.

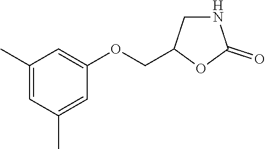

Metaxalone is a substrate of CYP1A2 and CYP2C19, an inhibitor of CYP1A2, CYP2B6, CYP2C9, CYP2C19, CYP2D6, CYP2E1, and CYP3A, and an inducer of CYP1A2 and CYP3A4.

Chemically, metaxalone is 5-[(3,5-dimethylphenoxy)methyl]-2-oxazolidinone. The formula is C₁₂H₁₅NO₃. The molecular weight is 221.25.

Alginic acid, also called algin, is a naturally occurring, edible polysaccharide found in brown algae. It is hydrophilic and forms a viscous gum when hydrated. With metals such as sodium and calcium, its salts are known as alginates. Its color ranges from white to yellowish-brown. It is sold in filamentous, granular, or powdered forms. Other names include Alginic acid; E400; [D-ManA(β1→4)L-GulA(α1→4)]n. The structure for alginic acid is shown below.

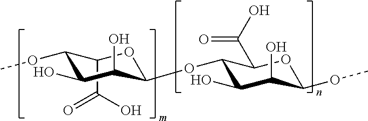

Commercial grade alginate are extracted from giant kelp Macrocystis pyrifera, Ascophyllum nodosum, and types of Laminaria. Alginates are also produced by two bacterial genera Pseudomonas and Azotobacter, which played a major role in the unraveling of its biosynthesis pathway. Bacterial alginates are useful for the production of micro- or nanostructures suitable for medical applications. Sodium alginate is the sodium salt of alginic acid. Sodium alginate is a gum. Potassium alginate is the potassium salt of alginic acid. Calcium alginate, is made from sodium alginate from which the sodium ion has been removed and replaced with calcium (ion exchange).

The manufacturing process used to extract sodium alginates from brown seaweed fall into two categories: 1) calcium alginate method and, 2) alginic acid method. Chemically the process is simple, but difficulties arise from the physical separations required between the slimy residues from viscous solutions and the separation of gelatinous precipitates that hold large amounts of liquid within their structure, so they resist filtration and centrifugation.

Alginate absorbs water quickly, which makes it useful as an additive in dehydrated products such as slimming aids, and in the manufacture of paper and textiles. It is also used for waterproofing and fireproofing fabrics, in the food industry as a thickening agent for drinks, ice cream, cosmetics, and as a gelling agent for jellies. Sodium alginate is mixed with soybean flour to make meat analogue.

Alginate is used as an ingredient in various pharmaceutical preparations, such as Gaviscon, in which it combines with bicarbonate to inhibit gastroesophageal reflux. Sodium alginate is used as an impression-making material in dentistry, prosthetics, lifecasting, and for creating positives for small-scale casting.

Propylene glycol alginate (PGA) is an emulsifier, stabilizer, and thickener used in food products. It is a food additive with E number E405. Chemically, propylene glycol alginate is an ester of alginic acid, which is derived from kelp. Some of the carboxyl groups are esterified with propylene glycol, some are neutralized with an appropriate alkali, and some remain free. Other names include: hydroxypropyl alginate, propane 1,2-diol alginate, and E405.

The molar mass is 234.21 per structural unit (theoretical). The appearance is white to yellowish brown filamentous, grainy, granular or powdered forms and is soluble in water.

The drug label for Metaxalone Tablets for oral use 640 mg (Revised February 2022) is found at https://www.accessdata.fda.gov/drugsatfdadocs/label/2022/022503s0011b1.pdf and is incorporated herein by reference in its entirety.

Recent evidence indicates that mitochondrial homeostasis is critical for myelination and maintenance of peripheral nerve function. Mice lacking the metabolic transcriptional coactivator peroxisome proliferator activated receptor γ coactivator 1α (PGC-1α) show reductions in expression of myelin-related proteins and exhibit myelin-associated lesions, so it was identified PGC-1α target genes in Schwann cells (SCs) in vitro to determine potential roles for PGC-1α in glia and tested whether PGC-1α was sufficient for SC differentiation and myelination. Forskolin-induced differentiation was associated with an upregulation of PGC-1α mRNA and protein, and while overexpression of PGC-1α upregulated genes such as manganese superoxide dismutase and estrogen-related receptor α, it was not sufficient for induction of differentiation. Both PGC-1α overexpression and forskolin exposure caused an increase in the mitochondrial fusion-related protein Mitofusin 1. These studies suggest that PGC-1α might be a potential target to promote mitochondrial stability during differentiation and myelination.

Recent evidence suggests that the maintenance of mitochondrial stability is critical for normal peripheral nerve function and neuronal-glial interactions. Charcot-Marie Tooth (CMT) disease, a devastating illness characterized by progressive peripheral nerve fiber deterioration, paralysis, and muscular atrophy, is frequently associated with mutations in genes controlling mitochondrial homeostasis (especially CMT2a). Mitochondrial homeostasis requires a delicate balance between fission and fusion, the disturbance of which can lead to decreased cellular respiration, decreased cell growth, and cell death. Mitochondrial fusion is orchestrated by several groups of proteins, including the transcription factor estrogen-related receptor α (ERRα) and the mitofusins (Mfn1, Mfn2), which regulate mitochondrial network formation, glucose and oxygen consumption, and mitochondrial membrane potential. The process of mitochondrial proliferation (biogenesis), on the other hand, is associated with increases in the transcription factors nuclear respiratory 1 (NRF-1) and nuclear respiratory factor 2 (GA-binding protein α and β) and the expression of proteins involved in mitochondrial DNA transcription and oxidative phosphorylation. The ERRα and NRF-1/NRF-2-dependent pathways function in parallel to influence mitochondrial gene expression and function.

A series of studies have demonstrated that the transcriptional coactivator peroxisome proliferator activated receptor γ coactivator 1α (PGC-1α) can regulate mitochondrial function, ERRα and NRF-1 activity, and mitofusin expression. Interestingly, myelin abnormalities have been observed in brains from mice lacking PGC-1α, but to date, there is no information about the roles of PGC-1α in glia. In the liver, PGC-1α expression is induced by glucocorticoids and agents that activate the cAMP/protein kinase A pathway, leading to the stimulation of gluconeogenesis via activation of the transcription factor CREB (cyclic AMP responsive element binding protein). In the peripheral nerve, the protein kinase A/CREB pathway is crucial for the differentiation of Schwann cells (SCs), so it is possible that PGC-1α is involved in SC differentiation.

In this study, it was hypothesized that differentiation of SCs by activation of the protein kinase A pathway involves the upregulation of PGC-1α and PGC-1α-target genes and sought to determine whether differentiation and/or mitochondrial pathways are regulated by PGC-1α in SCs. (See also, Cowell, Rita M et al. “Regulation of PGC-1 lpha and PGC-1 lpha-responsive genes with forskolin-induced Schwann cell differentiation.” Neuroscience letters vol. 439.3 (2008): 269-74).

Volumetric muscle loss (VML) injury is characterized by a non-recoverable loss of muscle fibers due to ablative surgery or severe orthopaedic trauma, that results in chronic functional impairments of the soft tissue. Currently, the effects of VML on the oxidative capacity and adaptability of the remaining injured muscle are unclear. A better understanding of this pathophysiology could significantly shape how VML-injured patients and clinicians approach regenerative medicine and rehabilitation following injury. Herein, the data indicated that VML-injured muscle has diminished mitochondrial content and function (i.e., oxidative capacity), loss of mitochondrial network organization, and attenuated oxidative adaptations to exercise. However, forced PGC-1α over-expression rescued the deficits in oxidative capacity and muscle strength. This implicates physiological activation of PGC1-α as a limiting factor in VML-injured muscle's adaptive capacity to exercise and provides a mechanistic target for regenerative rehabilitation approaches to address the skeletal muscle dysfunction.

Oxidative capacity is a cornerstone of skeletal muscle health, and for the past 40 years, it has been known that the most robust physiologic adaptation to regularly scheduled physical activity (i.e., exercise/overload training) is an increase in oxidative capacity. Improvements in muscle oxidative capacity are made possible with exercise training through adaptations affecting the density and function of the intramuscular mitochondrial network. The signaling pathways that initiate and coordinate mitochondrial improvements with exercise are complex, but advancements in molecular biology in the last two decades have revealed many of the key players involved. Most notably, the transcription factor PGC-1α (peroxisome proliferator-activated receptor gamma, coactivator 1 alpha) is considered a critical molecular modulator of skeletal muscle oxidative plasticity because it regulates gene expression patterns for expansion of the mitochondrial network (i.e., mitochondrial biogenesis), angiogenesis, and motor neuron associated adaptations with exercise training. Expansion of the vasculature and mitochondrial network with exercise training enhances the functional capacity of the muscle (e.g., fatigue resistance), and in general, this physiologic type of acclimation is considered beneficial for human performance and health.

Large-scale skeletal muscle trauma, such as volumetric muscle loss (VML) injury, is unique in that the muscle is not able to regenerate muscle fibers with endogenous repair systems and as a result, cannot fully recover strength. The loss of muscle function (i.e., contractility) can exceed the loss of tissue mass, and this permanent functional deficit leaves patients with lifelong disability for which there is currently no corrective physical rehabilitation guidelines. Furthermore, the extent to which the remaining skeletal muscle can adapt to rehabilitation is unclear. Recent preclinical work has investigated various models of ‘physical rehabilitation’ following VML in the forms of voluntary wheel running forced treadmill running, chronic-intermittent electrical nerve stimulation and/or passive range of motion exercises and collectively found modest contractile adaptations are possible. However, clinical reports have indicated that patients only see moderate improvements and then hit the ceiling where further physical therapy, no matter the type or intensity, does not result in an increase in function. Collectively, investigations of physical rehabilitation following VML have resulted in, at best, modest improvements in muscle function following VML injury without any physiological rationale or mechanistic understanding for the lack of significant response.

Overwhelmingly, investigations of preclinical outcome measures have focused on histologic and/or contractile aspects of the injured muscle with very few investigations focusing on any aspect of the oxidative plasticity of the remaining muscle. Notably, a reduced metabolic gene response (i.e., PGC-1α and SIRT-1) in VML-injured muscle following voluntary wheel running compared to uninjured muscle has been reported. Furthermore, mitochondrial respiration rates (measured from both the VML-injured and contralateral uninjured mouse muscles) were less than that of injury naïve mice suggesting a more chronic and systemic effect of VML injury on muscle oxidative capacity has been reported. Therefore, VML-injured patients may be susceptible to lifelong impairments in skeletal muscle oxidative capacity that can result in additional comorbidities as systemic reductions in skeletal muscle oxidative capacity are associated with an increased risk for a host of disorders such as diabetes and cardiovascular disease.

One apparent reason for the lack of investigation into oxidative capacity of VML-injured muscle is the difficulty in assessing physiological mechanisms of mitochondrial function. Herein, combined high-resolution mitochondrial respirometry was combined with mitochondrial enzyme kinetics and 2-photon microscopy to overcome this difficulty. Other work utilized 2-photon imaging to characterize the highly organized mitochondrial network and brought to light the importance of the network as it relates to the function of the mitochondria. An important innovation of the current work was the use of 2-photon microscopy to investigate changes in the structural integrity of the mitochondrial network as this aspect of mitochondrial physiology is likely disrupted following VML injury. The combination of all of these proven techniques has allowed for extensive characterization of multiple aspects of mitochondrial physiology in VML-injured muscle, which provides a new and unique prospective of the impacts of VML on mitochondria.

Repair of VML injury has been approached extensively by biomedical engineering and regenerative medicine experts over the past decades, yet no clear indication of clinically meaningful therapies capable of significantly improving skeletal muscle function have emerged. It is posited that a dysfunctional oxidative capacity contributes to a poor regenerative niche within the VML-injured muscle that ultimately limits regenerative approaches. Here, it is reported that the pathophysiology of the remaining muscle after VML includes widespread impairments in mitochondrial structure and respiratory function, and an insensitivity to physiological stimuli known to enhance mitochondrial structure and function (i.e., exercise). It is identified that the activation of the transcription factor PGC-1α as the limiting factor to exercise-induced adaptation and demonstrate the forced over-expression of PGC-1α rescue a substantial portion of the VML pathophysiology. (See also, Southern, W. M., et al. PGC-1α overexpression partially rescues impaired oxidative and contractile pathophysiology following volumetric muscle loss injury. Sci Rep 9, 4079 (2019)).

Skeletal muscle tissue has an enormous regenerative capacity that is instrumental for a successful defense against muscle injury and wasting. The peroxisome proliferator-activated receptor γ coactivator 1α (PGC-1α) exerts therapeutic effects in several muscle pathologies, but its role in damage-induced muscle regeneration is unclear.

PGC-1α exerts beneficial effects on muscle inflammation that might contribute to the therapeutic effects of elevated muscle PGC-1α in different models of muscle wasting. (See also, Dinulovic I, et al., PGC-1α modulates necrosis, inflammatory response, and fibrotic tissue formation in injured skeletal muscle. Skelet Muscle. 2016 Nov. 8; 6:38).

Mitochondrial degeneration is considered to play an important role in the development of diabetic peripheral neuropathy in humans. Mitochondrial degeneration and the corresponding protein regulation associated with the degeneration were studied in an animal model of diabetic neuropathy. PGC-1α and its-regulated transcription factors including TFAM and NRF-1, which are master regulators of mitochondrial biogenesis, are significantly downregulated in streptozotocin diabetic dorsal root ganglion (DRG) neurons. Diabetic mice develop peripheral neuropathy, loss of mitochondria, decreased mitochondrial DNA content and increased protein oxidation. Importantly, this phenotype is exacerbated in PGC-1α (−/−) diabetic mice, which develop a more severe neuropathy with reduced mitochondrial DNA and a further increase in protein oxidation. PGC-1α (−/−) diabetic mice develop an increase in total cholesterol and triglycerides, and a decrease in TFAM and NRF-1 protein levels.

Loss of PGC-1α causes severe mitochondrial degeneration with vacuolization in DRG neurons, coupled with reduced state 3 and 4 respiration, reduced expression of oxidative stress response genes and an increase in protein oxidation. In contrast, overexpression of PGC-1α in cultured adult mouse neurons prevents oxidative stress associated with increased glucose levels. The study provides new insights into the role of PGC-1α in mitochondrial regeneration in peripheral neurons and suggests that therapeutic modulation of PGC-1α function is an attractive approach for treatment of diabetic neuropathy.

Diabetes-induced-oxidative damage in neurons, axons, and Schwann cells has been proposed as a unifying mechanism for diabetic neuropathy. One mechanism for generation of oxidative stress is that an increased metabolic influx into mitochondria (Mt) increases respiration and results in a high proton gradient, leading to increased production of reactive oxygen species (ROS). Consistent with this notion, there is an increase in Mt inner membrane depolarization and degeneration of Mt in diabetic neuropathy. These impaired Mt may be rescued by the activation of Mt biogenesis or regeneration of new Mt. Thus, activation of Mt biogenesis may be protective under conditions where significant Mt degeneration is present.

Peroxisome proliferator-activated receptor-gamma co-activator 1α (PGC-1α) is a transcriptional co-activator and a master regulator for Mt biogenesis in many tissues including the nervous system. PGC-1α is a promising target for therapy for neurological disease. For example, the pan-PPAR agonist, bezafibrate, upregulates PGC-1α and exerts beneficial effects in a transgenic mouse model of Huntington's disease. PGC-1α activates transcriptional factors such as nuclear respiratory factor 1 (NRF-1) and Mt transcription factor A (TFAM), which in turn induce Mt respiration proteins and lead to replication of the Mt genome. PGC-1α has been mapped to chromosome 4P15.1, a region associated with basal insulin levels in Pima Indians that are at high risk of developing diabetes and diabetic related complications. Furthermore, common polymorphisms of PGC-1α are associated with conversion from impaired glucose tolerance to diabetes. Thus, it is likely that PGC-1α and its downstream signaling intermediates are important in normal Mt regulation in diabetic subjects.

PGC-1α has been clearly demonstrated in human brain and other neurons, and knockout of this gene in mice is associated with CNS neurodegeneration that includes the formation of large vacuoles in the neuropil and defects in energy metabolism. However, despite these findings in the CNS and other tissues, the roles of PGC-1α in Mt biogenesis in the peripheral nervous system (PNS) and specifically in diabetic neuropathy are unknown. Given the importance of PGC-1α in regulating Mt function of CNS neurons and evidence of downregulation of PGC-1α in diabetes, it was sought to determine whether loss of PGC-1α by genetic ablation or diabetic stress causes peripheral nervous system dysfunction and if overexpression of PGC-1α can prevent oxidative injury. It is hypothesized that impairment of PGC-1α mediated Mt regulation could contribute to the pathogenesis of diabetic neuropathy and that PGC-1α may serve as a target for treatment.

Neuropathic pain is a debilitating disease with few effective treatments. Emerging evidence indicates the involvement of mitochondrial dysfunction and oxidative stress in neuropathic pain. Nuclear factor erythroid 2-related factor 2 (NRF-2) is a potent regulator of the antioxidant response system. In this study, it was investigated whether RTA-408 (RTA), a synthetic triterpenoid under clinical investigation) could activate NRF-2 and promote mitochondrial biogenesis (MB) to reverse neuropathic pain and the underlying mechanisms.

Methods. Neuropathic pain was induced by chronic constriction injury (CCI) of the sciatic nerve. Pain behaviors were measured via the von Frey test and Hargreaves plantar test. The L4-6 spinal cord was collected to examine the activation of NRF-2 and MB. Results. RTA-408 treatment significantly reversed mechanical allodynia and thermal hyperalgesia in CCI mice in a dose-dependent manner. Furthermore, RTA-408 increased the activity of NRF-2 and significantly restored MB that was impaired in CCI mice in an NRF-2-dependent manner. Peroxisome proliferator-activated receptor-gamma coactivator-1 alpha (PGC-1α) is the key regulator of MB. It was found that the PGC-1α activator also induced a potent analgesic effect in CCI mice. Moreover, the antinociceptive effect of RTA-408 was reversed by the preinjection of the PGC-1α inhibitor.

Conclusions. NRF-2 activation attenuates chronic constriction injury-induced neuropathic pain via induction of PGC-1α-mediated mitochondrial biogenesis in the spinal cord. Our results indicate that NRF-2 may be a potential therapeutic strategy to ameliorate neuropathic pain and many other disorders with oxidative stress and mitochondrial dysfunction.

Neuropathic pain arises due to a primary lesion or dysfunction affecting the somatosensory nervous system, which markedly impairs the patients' quality of life and reduces individual productivity. Unfortunately, the efficacy of pharmacologic treatment is limited and is associated with side effects and risks of abuse. Therefore, identification of therapeutic strategies is considered to be a significant and unmet need.

Despite rapid advance over the past decades, the mechanisms underlying the development and maintenance of chronic pain remain to be elucidated. However, accumulating evidence indicates that oxidative stress and mitochondrial dysfunction are involved in various animal models of chronic pain. Mitochondrial biogenesis (MB) is the process of producing new functional mitochondria, which could restore mitochondrial function after various stimuli or injuries. The main regulatory factor of MB is peroxisome proliferator-activated receptor coactivator 1α (PGC-1α). PGC-1α displays its functions by increasing many transcription factors, including nuclear respiratory factors 1 and 2 (NRF-1 and NRF-2). NRF-1 and PGC-1α coactivate the transcriptional function of mitochondrial transcription factor A (TFAM) that directly promotes transcription and replication of the mitochondrial genome.

The antioxidant response element (ARE) is a DNA regulatory element, mainly binding with nuclear factor erythroid 2-related factor 2 (NRF-2) to activate these genes (such as heme oxygenase-1 (HO-1), NRF-1, and Hmox1). Under physiologic conditions, NRF-2 is sequestered to the cytoplasm by Kelch-like ECH-associated protein 1—(Keap1-) NRF-2 complex and ubiquitin degradation. However, oxidative stress triggers the dissociation of the Keap1-NRF-2 complex. NRF-2 then enters the nucleus and activates and regulate the transcription of antioxidant-related genes. NRF-2 is believed to be a master regulator of endogenous antioxidant defense and MB. The principal role of NRF-2/ARE in MB has been revealed by HO-1 activity in several models. Previous studies demonstrated that oltipraz and rosiglitazone significantly attenuate paclitaxel-induced neuropathic pain via induction of the NRF-2/HO-1 signaling pathway in the spinal cord. Moreover, several studies have illustrated that NRF-2 promoted MB via the regulation of PGC-1α. Thus, NRF-2 may be an attractive therapeutic target to stimulate MB under neuropathic pain conditions in a PGC-1α dependent manner.

RTA-408 (RTA), a novel NRF-2 activator, is a member of the synthetic oleanane triterpenoid compounds. It is currently being used in several clinical trials for the prevention and treatment of a variety of diseases, including radiation-induced dermatitis (NCT02142959), advanced solid cancers (melanoma or non-small-cell lung cancer) (NCT02029729), and Friedreich ataxia (NCT02255435). A previous study has shown that RTA-408 plays a critical role in mitigating radiation-induced bone marrow suppression by activating NRF-2. Notably, a recent study has demonstrated that RTA-408 could stimulate MB and benefit patients with mitochondrial myopathy (NCT02255422). Another study demonstrated that activation of NRF-2 with RTA-408 exerted a neuroprotective and disease-modifying effect by inhibiting reactive oxygen species production, mitochondrial depolarization, and neuronal death. Thus, this study investigated the promising effect of RTA-408 in the CCI model, and it is hypothesized that RTA-408 may exert analgesic effects via induction of PGC-1α-mediated MB in the spinal cord in a mice model of chronic constriction injury (CCI).

Impaired AMPK is associated with a wide spectrum of clinical and pathological conditions, ranging from obesity, altered responses to exercise or metabolic syndrome, to inflammation, disturbed mitochondrial biogenesis and defective response to energy stress. Fibromyalgia (FM) is a world-wide diffused musculoskeletal chronic pain condition that affects up to 5% of the general population and comprises all the above mentioned pathophysiological states. Here, the involvement of AMPK activation in fibroblasts derived from FM patients was tested. AMPK was not phosphorylated in fibroblasts from FM patients and was associated with decreased mitochondrial biogenesis, reduced oxygen consumption, decreased antioxidant enzymes expression levels and mitochondrial dysfunction.

However, mtDNA sequencing analysis did not show any important alterations which could justify the mitochondrial defects. AMPK activation in FM fibroblast was impaired in response to moderate oxidative stress. In contrast, AMPK activation by metformin or incubation with serum from caloric restricted mice improved the response to moderate oxidative stress and mitochondrial metabolism in FM fibroblasts. These results suggest that AMPK plays an essential role in FM pathophysiology and could represent the basis for a valuable new therapeutic target/strategy. Furthermore, both metformin and caloric restriction could be an interesting therapeutic approach in FM.

Restless legs syndrome (RLS) is a sensorimotor disorder characterized by extreme discomfort due to irresistible urges to move the legs. The symptoms worsen during rest, mainly at night, and the symptoms can be at least partially and temporarily relieved by physical activity. Parkinson's disease (PD) is one of the most common neurodegenerative diseases with motor symptoms and non-motor abnormalities. Various previous studies have found that the prevalence rate of RLS was higher in PD patients than in the general population, affecting 10-50% of parkinsonian patients. Several studies have also suggested that RLS is a possible preclinical marker of PD. Indeed, most genetics and pathology studies point to a strong association between RLS and PD.

Although the etiology of PD patients with RLS is still poorly understood, pathophysiological and pharmacotherapeutical studies suggest that dopamine (DA) dysfunction plays an important role in both PD and RLS pathophysiology. Brain-derived neurotrophic factor (BDNF) is a critical member of the neurotrophin family, which is involved in the mediation of the neuronal survival and development. It is a protein mostly expressed in the central nervous system, especially in the striatum, substantia nigra, and ventral tegmental area, which contains a major portion of the dopaminergic (DAergic) cell groups of the ventral midbrain. Several lines of evidence reveal that the pleiotropic activities of BDNF play a vital role in the survival and maintenance of DAergic neurons, and lower BDNF expression in substantia nigra is associated with dopamine neuronal loss in PD patients compared with that in controls. Consistently, a lot of improving treatments of PD are accompanied by BDNF enhancement. These studies suggest that the reduced BDNF levels may be associated with pathological alterations of the DAergic neurons in the substantia nigra.

A goal of this study was to explore the association between BDNF and RLS in PD patients. To achieve this goal, a comparatively large sample of PD patients (n=249) were recruited and matched controls (n=326), and tried to establish correlations between serum BDNF levels and RLS assessed by the International Restless Legs Syndrome Study Group Rating Scale (IRLSSG-RS). It is hypothesized that serum BDNF levels would be decreased in PD patients with RLS, and reduced BDNF levels might be associated with the severity of RLS in PD.

Fibromyalgia syndrome (FMS) is considered a musculoskeletal disorder associated to other symptoms including chronic pain. Since the hypothesis of FMS etiogenesis is consistent with mitochondrial dysfunction and oxidative stress, the pathophysiological correlation among these factors were analyzed while studying some proteins involved in the mitochondrial homeostasis. Attention was focused on the roles of peroxisome proliferator activated receptor gamma coactivator-1 alpha (PGC-1α), mitofusin2 (Mfn2), and coenzyme Q10 (CoQ10) in reserpine-induced myalgic (RIM) rats that manifest fibromyalgia-like chronic pain symptoms. First, RIM rats are a good model for studying the pathophysiology of FMS and moreover, it was found that PGC-1α, Mfn2, and CoQ10 are involved in FMS. In fact, their expressions were reduced in gastrocnemius muscle determining an incorrect mitochondrial homeostasis.

Presently, none of the currently available drugs are fully effective against the symptoms of this disease and they, often, induce several adverse events; hence, many scientists have taken on the challenge of searching for non-pharmacological treatments. Another goal of this study was therefore the evaluation of the potential benefits of melatonin, an endogenous indoleamine having several functions including its potent capacity to induce antioxidant enzymes and to determine the protective or reparative mechanisms in the cells. It was observed that melatonin supplementation significantly preserved all the studied parameters, counteracting oxidative stress in RIM rats and confirming that this indoleamine should be taken in consideration for improving health and/or counteract mitochondrial related diseases.

Chronic fatigue syndrome (CFS) and fibromyalgia (FM) are complex and serious illnesses that affect approximately 2.5% and 5% of the general population worldwide, respectively. The etiology is unknown; however, recent studies suggest that mitochondrial dysfunction has been involved in the pathophysiology of both conditions. It has been have investigated the possible association between mitochondrial biogenesis and oxidative stress in patients with CFS and FM. 23 CFS patients were studied, 20 FM patients, and 15 healthy controls. Peripheral blood mononuclear cell showed decreased levels of Coenzyme Q10 from CFS patients (p<0.001 compared with controls) and from FM subjects (p<0.001 compared with controls) and ATP levels for CFS patients (p<0.001 compared with controls) and for FM subjects (p<0.001 compared with controls).

On the contrary, CFS/FM patients had significantly increased levels of lipid peroxidation, respectively (p<0.001 for both CFS and FM patients with regard to controls) that were indicative of oxidative stress-induced damage. Mitochondrial citrate synthase activity was significantly lower in FM patients (p<0.001) and, however, in CFS, it resulted in similar levels than controls. Mitochondrial DNA content (mtDNA/gDNA ratio) was normal in CFS and reduced in FM patients versus healthy controls, respectively (p<0.001). Expression levels of peroxisome proliferator-activated receptor gamma-coactivator 1-alpha and transcription factor A, mitochondrial by immunoblotting were significantly lower in FM patients (p<0.001) and were normal in CFS subjects compared with healthy controls. These data lead to the hypothesis that mitochondrial dysfunction-dependent events could be a marker of differentiation between CFS and FM, indicating the mitochondria as a new potential therapeutic target for these conditions.

Chemotherapy-induced neuropathic pain is a debilitating and common side effect of cancer treatment and so far no effective drug is available for treatment of the serious side effect. Previous studies have demonstrated μ2-adrenoreceptor (ADRB2) agonists can attenuate neuropathic pain. However, the role of ADRB2 in paclitaxel-induced neuropathic pain (PINP) remains unclear. In this study, the effect of formoterol was investigated, a long-acting ADRB2 agonist, and related mechanisms in PINP. A rat model of PINP was established by intraperitoneal injection of paclitaxel (2 mg/kg) every other day with a final cumulative dose of 8 mg/kg. Hind paw withdrawal thresholds (PWTs) in response to von Frey filament stimuli were used to evaluate mechanical allodynia. Western blot was used to examine the expression of ADRB2, peroxisome proliferator-activated receptor coactivator-1α (PGC-1α), nuclear respiratory factors 1 (NRF-1) and mitochondrial transcription factor A (TFAM) and the immunofluorescence was to detect the cellular localization of ADRB2 and PGC-1α in the spinal cord.

Moreover, mitochondrial DNA (mtDNA) copy number by qPCR was measured. In our study, formoterol attenuated established PINP and delayed the onset of PINP. Formoterol restored ADRB2 expression as well as mtDNA copy number and PGC-1α, NRF-1, and TFAM protein expression, which are major genes involved in mitochondrial biogenesis, in the spinal cord of PINP rats. Moreover, it was found that the analgesic effect of formoterol against PINP was partially abolished by PGC-1α inhibitor SR-18292. Collectively, these results demonstrated the activation of ADRB2 with formoterol ameliorates PINP at least partially through induction of mitochondrial biogenesis.

Chronic fatigue syndrome (CFS) and fibromyalgia (FM) are complex and serious illnesses that affect approximately 2.5% and 5% of the general population worldwide, respectively. The etiology is unknown; however, recent studies suggest that mitochondrial dysfunction has been involved in the pathophysiology of both conditions. The possible association between mitochondrial biogenesis and oxidative stress was investigated in patients with CFS and FM. 23 CFS patients were studied, 20 FM patients, and 15 healthy controls. Peripheral blood mononuclear cell showed decreased levels of Coenzyme Q10 from CFS patients (p<0.001 compared with controls) and from FM subjects (p<0.001 compared with controls) and ATP levels for CFS patients (p<0.001 compared with controls) and for FM subjects (p<0.001 compared with controls).

On the contrary, CFS/FM patients had significantly increased levels of lipid peroxidation, respectively (p<0.001 for both CFS and FM patients with regard to controls) that were indicative of oxidative stress-induced damage. Mitochondrial citrate synthase activity was significantly lower in FM patients (p<0.001) and, however, in CFS, it resulted in similar levels than controls. Mitochondrial DNA content (mtDNA/gDNA ratio) was normal in CFS and reduced in FM patients versus healthy controls, respectively (p<0.001). Expression levels of peroxisome proliferator-activated receptor gamma-coactivator 1-alpha and transcription factor A, mitochondrial by immunoblotting were significantly lower in FM patients (p<0.001) and were normal in CFS subjects compared with healthy controls. These data lead to the hypothesis that mitochondrial dysfunction-dependent events could be a marker of differentiation between CFS and FM, indicating the mitochondria as a new potential therapeutic target for these conditions.

Chemotherapy-induced neuropathic pain is a debilitating and common side effect of cancer treatment and so far no effective drug is available for treatment of the serious side effect. Previous studies have demonstrated β2-adrenoreceptor (ADRB2) agonists can attenuate neuropathic pain. However, the role of ADRB2 in paclitaxel-induced neuropathic pain (PINP) remains unclear. In this study, the effect of formoterol was investigated, a long-acting ADRB2 agonist, and related mechanisms in PINP. A rat model of PINP was established by intraperitoneal injection of paclitaxel (2 mg/kg) every other day with a final cumulative dose of 8 mg/kg. Hind paw withdrawal thresholds (PWTs) in response to von Frey filament stimuli were used to evaluate mechanical allodynia. Western blot was used to examine the expression of ADRB2, peroxisome proliferator-activated receptor coactivator-1α (PGC-1α), nuclear respiratory factors 1 (NRF-1) and mitochondrial transcription factor A (TFAM) and the immunofluorescence was to detect the cellular localization of ADRB2 and PGC-1α in the spinal cord.

Moreover, mitochondrial DNA (mtDNA) copy number was measured by qPCR. In our study, formoterol attenuated established PINP and delayed the onset of PINP. Formoterol restored ADRB2 expression as well as mtDNA copy number and PGC-1α, NRF-1, and TFAM protein expression, which are major genes involved in mitochondrial biogenesis, in the spinal cord of PINP rats. Moreover, it was found that the analgesic effect of formoterol against PINP was partially abolished by PGC-1α inhibitor SR-18292. Collectively, these results demonstrated the activation of ADRB2 with formoterol ameliorates PINP at least partially through induction of mitochondrial biogenesis.

Oxidative stress can be induced by various stimuli and altered in certain conditions, including exercise and pain. Although many studies have investigated oxidative stress in relation to either exercise or pain, the literature presents conflicting results. Therefore, this review critically discusses existing literature about this topic, aiming to provide a clear overview of known interactions between oxidative stress, exercise, and pain in healthy people as well as in people with chronic pain, and to highlight possible confounding factors to keep in mind when reflecting on these interactions. In addition, autonomic regulation and epigenetic mechanisms are proposed as potential mechanisms of action underlying the interplay between oxidative stress, exercise, and pain. This review highlights that the relation between oxidative stress, exercise, and pain is poorly understood and not straightforward, as it is dependent on the characteristics of exercise, but also on which population is investigated. To be able to compare studies on this topic, strict guidelines should be developed to limit the effect of several confounding factors. This way, the true interplay between oxidative stress, exercise, and pain, and the underlying mechanisms of action can be revealed and validated via independent studies.

### Additional Embodiments

[Embodiment 1] A method of treating chronic or acute conditions associated with specific or non-specific neuroinflammation in a human consisting of inhibiting the MAO-A isoform enzymatic pathway by administering an oral dosage form comprising:
a therapeutic dose of first and second particulate metaxalones in one or more suitable forms selected from salts, co-crystals, solvates, free form, enantiomers, and polymorphs thereof, and,
one or more pharmaceutically suitable excipients, wherein the therapeutic dose optionally achieves a stable dissolution profile over time.

[Embodiment 2] A method of inhibiting the MAO-A enzymatic pathway consisting of administering an oral dosage form comprising:
a therapeutic dose of first and second particulate metaxalones in one or more suitable forms selected from salts, co-crystals, solvates, free form, enantiomers, and polymorphs thereof, and,
one or more pharmaceutically suitable excipients, wherein the therapeutic dose optionally achieves a stable dissolution profile over time.

[Embodiment 3] A method of treating chronic or acute conditions associated with specific or non-specific neuroinflammation in a human consisting of administering an oral dosage form comprising:
a therapeutic dose of first and second particulate metaxalones in one or more suitable forms selected from salts, co-crystals, solvates, free form, enantiomers, and polymorphs thereof, and,
one or more pharmaceutically suitable excipients, wherein the therapeutic dose optionally achieves a stable dissolution profile over time.

[Embodiment 4] A method of restoring or increasing IL-13 and inhibiting STAT3 consisting of administering an oral dosage form comprising:
a therapeutic dose of first and second particulate metaxalones in one or more suitable forms selected from salts, co-crystals, solvates, free form, enantiomers, and polymorphs thereof, and,
one or more pharmaceutically suitable excipients, wherein the therapeutic dose optionally achieves a stable dissolution profile over time.

[Embodiment 5] The method of embodiment 4, further consisting of increasing white matter growth and development in the central nervous system by increasing and/or attenuating the natural pathways responsible for proper white matter cellular growth and repair.

[Embodiment 6] The method of embodiment 5, further consisting of treating demyelination disorders or diseases.

[Embodiment 7] A method of increasing or restoring PGC-1α consisting of administering an oral dosage form comprising:
a therapeutic dose of first and second particulate metaxalones in one or more suitable forms selected from salts, co-crystals, solvates, free form, enantiomers, and polymorphs thereof, and,
one or more pharmaceutically suitable excipients, wherein the therapeutic dose optionally achieves a stable dissolution profile over time.

[Embodiment 8] The method of embodiment 7, further consisting of treating one or more disorders or diseases selected from sciatica, fibromyalgia, neuropathic pain, ischemic stroke and traumatic brain injury.

[Embodiment 9] The method of embodiment 8, wherein said neuropathic pain is selected from mechanical allodynia, thermal hyperalgesia, diabetic neuropathy, chemotherapy neuropathy and post-operative pain.

[Embodiment 10] The method of embodiment 7, further consisting of restoring mitochondrial biogenesis.

[Embodiment 11] The method of embodiment 7, further consisting of stimulating NRF-1 and NRF-2.

[Embodiment 12] The method of embodiment 7, further consisting of preventing chronification of neuropathic pain due to haploinsufficiency.

[Embodiment 13] The method of embodiment 7, further consisting of stimulating AMPK.

[Embodiment 14] The method of embodiment 13, further consisting of bolstering gene expression and protein syntheses of MnSOD and catalase.

[Embodiment 15] The method of embodiment 13, further consisting of treating cardiovascular disorder or disease and treating vascular leakage caused by permeability.

[Embodiment 16] The method of embodiment 7, 13 or 14, further consisting of treating pain caused by nerve injury.

[Embodiment 17] The method of embodiment 7, 13 or 14, further consisting of preventing pain chronification caused by burn injury.

[Embodiment 18] A method of treating chronic venous insufficiency and increasing AMPK and PGC-1α and restoring MnSOD and catalase consisting of administering an oral dosage form comprising:
a therapeutic dose of first and second particulate metaxalones in one or more suitable forms selected from salts, co-crystals, solvates, free form, enantiomers, and polymorphs thereof, and,
one or more pharmaceutically suitable excipients, wherein the therapeutic dose optionally achieves a stable dissolution profile over time.

[Embodiment 19] The method of embodiment 7, 13, 14 or 18 further consisting of inhibiting NF-κB.

[Embodiment 20] The method of embodiment 7, 13, 14 or 18, further consisting of revitalizing muscle strength.

[Embodiment 21] The method of embodiment 7, 13 or 14, further consisting of initiating regeneration of muscular tissue.

[Embodiment 22] The method of embodiment 7, 13, 14 or 18, further consisting of promoting an antifibrotic state.

[Embodiment 23] The method of embodiment 7, 13 14 or 18, further consisting of preventing carpal tunnel fibrosis.

[Embodiment 24] The method of any one of embodiments 1-15 and 18, wherein said excipients are selected from alginic acid, propylene glycol alginate and combinations thereof.

[Embodiment 25] The method of any one of embodiments 1-15 and 18, comprising a dose in the range of 600-700 mg of metaxalone.

[Embodiment 26] The method of any one of embodiments 1-15 and 18, comprising ˜640 mg of metaxalone.

[Embodiment 27] The method of any one of embodiments 1-15 and 18, comprising:
40-80% wt of a first metaxalone and 20-60% wt of a second metaxalone,
wherein ≥90% wt of the first metaxalone has a particle size of ˜7-86μ, and,
wherein ≥35% wt of the second metaxalone is has a particle size in the range of ˜98 to 516μ.

[Embodiment 28] The method of any one of embodiments 1-15 and 18, wherein the chronic or acute, specific or non-specific neuroinflammatory condition is diabetic neuropathy.

[Embodiment 29] The method of any one of embodiments 1-15 and 18, wherein the chronic or acute, specific or non-specific, neuroinflammatory condition is fibromyalgia or back pain.

[Embodiment 30] The method of any one of embodiments 1-15 and 18, furthering consisting of substantially inducing or restoring homeostasis.

[Embodiment 31] The method of embodiments 1-15 and 18, further consisting of substantially restoring oxidative homeostasis.

[Embodiment 32] The method of embodiments 1-15 and 18, further consisting of substantially restoring basal phenotype from a neuroinflammatory state.

[Embodiment 33] The method of embodiments 1-15 and 18, further consisting of treating chronic pain induced depression.

[Embodiment 34] The method of embodiments 1-15 and 18, further consisting of treating restless leg syndrome.

[Embodiment 35] The method of embodiments 1-15 and 18, further consisting of improving neuroplasticity.

[Embodiment 36] The method of embodiments 1-15 and 18, further consisting of treating pain induced depression.

[Embodiment 37] The method of embodiments 1-15 and 18, further consisting of treating anxiety and mood disorder.

[Embodiment 38] The method of embodiments 1-15 and 18, further consisting of treating peripheral neuropathy.

[Embodiment 39] The method of embodiments 1-15 and 18, further consisting of treating herpetic neuralgia.

[Embodiment 40] The method of any one of embodiments 1-15 and 18, wherein said first and second metaxalone are crystalline racemic mixture.

[Embodiment 41] The method of any one of embodiments 1-15 and 18, wherein the oral dosage form induces a pharmacokinetic profile in adult males, under fasted conditions, of Tₘₐₓ˜1.5-12 hr, mean Cₘₐₓ˜2 mcg/ml, AUC˜16 mcg·h/ml, and T_{1/2}˜5 hr, and wherein said AUC0→∞ and mean Cₘₐₓ in adult females, under fasted conditions, is ˜40% greater as compared to adult males.

[Embodiment 42] The method of any one of embodiments 1-15 and 18, wherein the oral dosage form is administered without food.

[Embodiment 43] The method of any one of embodiments 1-15 and 18, wherein the oral dosage form is co-administered with food.

[Embodiment 44] The method of embodiment 43, wherein the food effect of high fat meal is ˜23% increase in Cₘₐₓ, no change in AUC0→∞, and Tₘₐₓ˜3.5˜24 hr.

[Embodiment 45] The method of embodiment 41, wherein, for fasted adult males, Tₘₐₓ˜3.0 hr.

[Embodiment 46] The method of embodiment 41, wherein Tₘₐₓ˜8 hr.

[Embodiment 47] The method of any one of embodiments 1-23, wherein the oral dosage form is administered 3 or 4 times daily.

[Embodiment 48] The method of any of the foregoing embodiments, wherein the dosage form comprises 640 mg of metaxalone.

[Embodiment 49] The method of any of the foregoing embodiments, wherein the dosage form has a stable dissolution profile over time.

### EXAMPLES

### Example 1

Experimental and clinical studies have suggested that several neurological disorders are associated with the occurrence of central nervous system neuroinflammation. Metaxalone is an FDA-approved muscle relaxant that has been reported to inhibit monoamine oxidase A (MAO-A). The aim of this study was to investigate whether metaxalone might exert antioxidant and anti-inflammatory effects in HMC3 microglial cells. An inflammatory phenotype was induced in HMC3 microglial cells through stimulation with interleukin-1β (IL-1β). Control cells and IL-1β-stimulated cells were subsequently treated with metaxalone (10, 20, and 40 μM) for six hours. IL-1β stimulated the release of the pro-inflammatory cytokines tumor necrosis factor-alpha (TNF-α) and interleukin-6 (IL-6), but reduced the anti-inflammatory cytokine interleukin-13 (IL-13).

The upstream signal consisted of an increased priming of nuclear factor-kB (NF-kB), blunted peroxisome proliferator-activated receptor gamma (PPARγ), and peroxisome proliferator-activated receptor gamma coactivator 1-alpha (PGC-1α) expression. IL-1β also augmented MAO-A expression/activity and malonaldehyde levels and decreased NRF-2 mRNA expression and protein levels. Metaxalone decreased MAO-A activity and expression, reduced NF-kB, TNF-α, and IL-6, enhanced IL-13, and also increased PPARγ, PGC-1α, and NRF-2 expression. The present experimental study suggests that metaxalone has potential for the treatment of several neurological disorders associated with neuroinflammation.

Several neurological diseases are associated with neuroinflammation that represents a rational target of therapy and, recently, a marked microglia inflammation has been associated with fibromyalgia. Fibromyalgia (FM) is a poorly understood musculoskeletal disorder characterized by chronic widespread pain accompanied by fatigue, sleep/mood disturbances, and cognitive dysfunction. (See, Clauw, D. J. Fibromyalgia: A clinical review. JAMA 2014, 311, 1547-1555). After osteoarthritis, fibromyalgia is considered the second most common rheumatic disorder with a higher prevalence in women than in men. (See, Vincent, A. et al, Prevalence of fibromyalgia: A population-based study in Olmsted County, Minnesota, utilizing the Rochester Epidemiology Project. Arthritis Care Res. 2013, 65, 786-792). Both its etiology and pathophysiology are still not fully known but central mechanisms are strongly involved, with an impairment of the central nervous system (CNS) in addition to the peripheral one. (See, Albrecht, D. S. et al, Differential dopamine function in fibromyalgia. Brain Imaging Behav. 2016, 10, 829-839). (See, Oaklander, A. L. et al., Objective evidence that small-fiber polyneuropathy underlies some illnesses currently labeled as fibromyalgia. Pain 2013, 154, 2310-2316).

The pain associated with fibromyalgia is related to the modulation and activation of different neural networks, including neurotransmitters, hormones, neuropeptides, cytokines, and chemokines that, working together, amplify pain perception. (See, Littlejohn, G. et al., Neurogenic inflammation in fibromyalgia. Semin. Immunopathol. 2018, 40, 291-300). This complex interactive mechanism is integrated in a tangle that involves the brain, spinal cord, and the peripheral tissues, which contributes to neurogenic inflammation. Patients affected by FM showed increased levels of neuropeptides and cytokines in their cerebrospinal fluid (CSF), such as substance P (SP) and interleukin-8 (IL-8) (See, Vaeroy, H. et al., Elevated CSF levels of substance P and high incidence of Raynaud phenomenon in patients with fibromyalgia: New features for diagnosis. Pain 1988, 32, 21-26)(See, Russell, I. J. et al., Elevated cerebrospinal fluid levels of substance P in patients with the fibromyalgia syndrome. Arthritis Rheum. 1994, 37, 1593-1601); the release of neuropeptides and pro-inflammatory cytokines orchestrates neuroinflammation.

The levels of IL-1, IL-6, IL-8, and TNF-α were found to also be increased in the blood of patients with fibromyalgia (See, Rodriguez-Pinto, I., et al., Fibromyalgia and cytokines. Immunol. Lett. 2014, 161, 200-203)(See also, Generaal, E. et al., Basal inflammation and innate immune response in chronic multisite musculoskeletal pain. Pain 2014, 155, 1605-1612)(See also, Uceyler, N. et al., Systematic review with meta-analysis: Cytokines in fibromyalgia syndrome. BMC Musculoskelet Disord. 2011, 12, 245): an association between IL-8, IL-6 and the severity of the disease has been demonstrated, thus showing that the release of pro-inflammatory cytokines negatively links to fibromyalgia and its prognosis. (See, Mendieta, D. et al., IL-8 and IL-6 primarily mediate the inflammatory response in fibromyalgia patients. J. Neuroimmunol. 2016, 290, 22-25).

Pregabalin, duloxetine, and milnacipran are approved in the US for the treatment of fibromyalgia and neuropathic pain, but their efficacy, safety, and compliance are not satisfactory. (See, Gota, C. E. What you can do for your fibromyalgia patient. Cleve. Clin. J. Med. 2018, 85, 367-376) (See also, Whiteside, N. et al., Proportion of contextual effects in the treatment of fibromyalgia-a meta-analysis of randomised controlled trials. Clin. Rheumatol. 2018, 37, 1375-1382). One of the priorities of the treatment of FM is pain management. In this context, muscle relaxants are drugs whose mechanism of action is based on the reduction of muscle spasm, and although their use is not convincing, muscle relaxants are approved and well accepted in clinical practice as therapeutic adjuvants for the treatment of chronic musculoskeletal pain. (See, Richards, B. L. et al., Muscle relaxants for pain management in rheumatoid arthritis. Cochrane Database Syst. Rev. 2012, 1, CD008922).

Metaxalone is a muscle relaxant, approved by the Food and Drug Administration as adjuvant therapy for the management of acute and painful musculoskeletal conditions, but it has no direct muscle relaxant effects. (See, Bosak, A. R. et al., Serotonin syndrome associated with metaxalone overdose. J. Med. Toxicol. 2014, 10, 402-405). The exact mechanism of action is not yet fully described, but it has been proposed as an inhibitor of monoamine oxidase MAO-A. (See, Cherrington, B. et al., Monoamine oxidase A inhibition by toxic concentrations of metaxalone. Clin. Toxicol. 2020, 58, 383-387). Currently, the MAO-inflammation connection is not fully elucidated; nevertheless, in a previous study, it was shown that co-exposure of cardiomyocytes to the pro-inflammatory cytokine IL-1β elicited MAO-related oxidative stress with subsequent mitochondrial dysfunction and endoplasmic reticulum stress. (See, Deshwal, S. et al. Monoamine oxidase-dependent endoplasmic reticulum-mitochondria dysfunction and mast cell degranulation lead to adverse cardiac remodeling in diabetes. Cell Death Differ. 2018, 25, 1671-1685).

Moreover, it has been reported that lipopolysaccharide (LPS) stimulation resulted in the upregulation of both MAO isoforms via a signal transduction pathway that appears to involve NF-κB. (See, Raţiu, C. et al., Monoamine oxidase inhibition improves vascular function and reduces oxidative stress in rats with lipopolysaccharide-induced inflammation. Gen. Physiol. Biophys. 2018, 37, 687-694).

Furthermore, MAO up-regulation triggered by LPS was observed in a rat periodontal disease model and treatment with a MAO inhibitor, phenelzine, was able to significantly reduce oxidative stress. (See, Ekuni, D. et al. Lipopolysaccharide-induced epithelial monoamine oxidase mediates alveolar bone loss in a rat chronic wound model. Am. J. Pathol. 2009, 175, 1398-1409).

Another recent study showed that moclobemide, a reversible MAO-A inhibitor, was able to attenuate the inflammatory process in a rat model of ischemia-reperfusion injury. (See, Vuohelainen, V. et al., Inhibition of monoamine oxidase A increases recovery after experimental cardiac arrest. Interact Cardiovasc. Thorac Surg. 2015, 21, 441-449). Additionally, it has been reported that MAO-A is involved in ROS generation in alternatively activated monocytes/macrophages. (See, Cathcart, M. K. et al., Monoamine oxidase A (MAO-A): A signature marker of alternatively activated monocytes/macrophages. Inflamm. Cell Signal. 2014, 1, e161)(See also, Bhattacharjee, A. et al., IL-4 and IL-13 employ discrete signaling pathways for target gene expression in alternatively activated monocytes/macrophages. Free Radic. Biol. Med. 2013, 54, 1-16).

All these previous works demonstrated that MAO-A inhibition was able to decrease ROS generation and the ROS-activated inflammatory process in particular through NF-κB pathway inhibition. Several studies showed that inflammatory cytokines in fibromyalgia patients could drive disturbances in neural networks during the interaction of the nervous system with immune cells, which eventually could lead to an increase in central and peripheral sensitization as well as neuroinflammation. (See, O'Mahony, L. F. et al., Is fibromyalgia associated with a unique cytokine profile? Rheumatology 2021, 12, 146).

For all these reasons, the aim of this study was to investigate the therapeutic potential of metaxalone in an in vitro experimental paradigm of microglial cell inflammation (obtained through IL-1β stimulation)(See, Hankittichai, P. et al., Oxyresveratrol Inhibits IL-1β-Induced Inflammation via Suppressing AKT and ERK1/2 Activation in Human Microglia, HMC3. Int. J. Mol. Sci. 2020, 21, 6054) that mimics the phenomenon of neuroinflammation involved in several neurological disorders, including fibromyalgia.

Several therapeutic approaches for the treatment of neurological diseases characterized by neuroinflammation, like fibromyalgia, have been proposed. (See, Abeles, M. et al., Update on fibromyalgia therapy. Am. J. Med. 2008, 121, 555-561). However, the rate of therapeutic failure with the available drugs is still very high, and this justifies the exploration of innovative treatments eventually emerging from robust preclinical data on the underlying physiopathology.

Recently, a new scenario has caught researchers' interest: neuroinflammation in the CNS may play an important role in amplifying pain perception and the correlated depression symptoms. (See, Kadetoff, D. et al., Evidence of central inflammation in fibromyalgia-increased cerebrospinal fluid interleukin-8 levels. J. Neuroimmunol. 2012, 242, 33-38). An increase in pro-inflammatory chemokine and cytokine levels would cause a sensitization of central nociceptors, causing negative effects on symptoms and worsening the prognosis of the affected patients. In fact, patients with fibromyalgia showed an increase in interleukin and TNF-α levels in serum and plasma, which contribute not only to the exacerbation of inflammatory response but also to pain.

Microglial cells are resident macrophages responsible for the mechanisms of defense and repair of nerve tissue. (See, Harry, G. J. et al., Microglia in the developing brain: A potential target with lifetime effects. Neurotoxicology 2012, 33, 191-206). However, these cells, once activated, can contribute to neuroinflammation and especially to the development of numerous neurodegenerative diseases. In fact, microglial cells release and activate several molecules involved in inflammatory processes such as iNOS, IL-1β, TNF-α, COX-1, COX-2, reactive oxygen species (ROS), and potentially neurotoxic compounds that cause neuronal dysfunctions and cell death. (See, Park, S. E. et al., Kaemferol acts through mitogen-activated protein kinases and protein kinase B/AKT to elicit protection in a model of neuroinflammation in BV2 microglial cells. Br. J. Pharmacol. 2011, 164, 3008-3025).

Therefore, despite the activation of microglia and neuroinflammation they might have a neuroprotective role; on the other hand, an exaggerated activation, resulting in a storm of pro-inflammatory cytokines, can contribute to the onset of neurological symptoms and neurodegenerative processes. (See, Lyman, M. et al., Neuroinflammation: The role and consequences. Neurosci. Res. 2013, 79, 1-12)(See also, Shabab, T. et al., Neuroinflammation pathways: A general review. Int. J. Neurosci. 2017, 127, 624-633). This also holds true for fibromyalgia; therefore, a possible therapeutic approach could aim at inhibiting the pro-inflammatory mediators produced by microglia.

Metaxalone (5-((3,5-dimethylphenoxy)methyl)-2-oxazolidinone) is a muscle relaxant indicated for the treatment of acute musculoskeletal pain, and even if its mechanism of action is still not fully known, a general sedation of the nervous system seems to be involved. (See, Vuletić, L. et al., Development of a Clinically Relevant Dissolution Method for Metaxalone Immediate Release Formulations Based on an IVIVC Model. Pharm. Res. 2018, 35, 163). Metaxalone has been approved by the Food and Drug Administration (FDA) as an adjuvant therapy in the treatment of acute and painful musculoskeletal conditions, and is commonly prescribed as a muscle relaxant, although it has no direct muscle relaxant effects.

However, it has been proposed that it may inhibit monoamine oxidase (MAO-A); this enzyme, besides being involved in the metabolism of serotonin, has been linked to several pathological conditions characterized by exaggerated oxidative stress and inflammation (See, Schwartz, T. L. A neuroscientific update on monoamine oxidase and its inhibitors. CNS Spectr. 2013, 18, 25-32)(See, Bianchi, P. et al. Oxidative stress by monoamine oxidase mediates receptor-independent cardiomyocyte apoptosis by serotonin and postischemic myocardial injury. Circulation 2005, 112, 3297-3305) therefore, its inhibition may represent a strategy to design anti-inflammatory drugs.

A recent report has unmasked the ability of metaxalone to exert anti-inflammatory activity; in fact, the drug caused a robust reduction in several inflammatory cytokines in LPS-stimulated mouse macrophages. (See, Yamaguchi, M. et al., Metaxalone Suppresses Production of Inflammatory Cytokines Associated with Painful Conditions in Mouse Macrophages RAW264.7 cells in Vitro: Synergistic Effect with β-caryophyllene. Curr. Mol. Med. 2020, 20, 643-652). However, this preliminary report used a cell line that has a scarce translational potential and did not address any attempt to dissect out the underlying mode/mechanism of action.

In the present study, metaxalone's anti-inflammatory effect was confirmed in microglial cells, the true resident macrophages of the CNS. Metaxalone suppressed the inflammatory phenotype triggered by IL-1β in a dose-dependent manner. Interestingly, the upstream signals that might be interrupted by metaxalonewere investigated. The drug inhibited the augmented expression of NF-kB and increased the reduced expression of PPARγ in microglia cells. Indeed, activation of this nuclear receptor has been linked to a strong down-regulation of the inflammatory phenotype in microglial cells. (See, Zhou, D. et al., TSPO Modulates IL-4-induced Microglia/Macrophage M2 Polarization via PPAR-γ Pathway. J. Mol. Neurosci. 2020, 70, 542-549)(See also, Sarathlal, K. C. et al., Exploring the Neuroprotective Potential of Rosiglitazone Embedded Nanocarrier System on Streptozotocin Induced Mice Model of Alzheimer's Disease. Nurotox. Res. 2021, 39, 240-255)(See also, Beheshti, F. et al., The effects of PPAR-γ agonist pioglitazone on hippocampal cytokines, brain-derived neurotrophic factor, memory impairment, and oxidative stress status in lipopolysaccharide-treated rats. Iran. J. Basic. Med. Sci. 2019, 22, 940-948). Therefore, our hypothesis is that metaxalone finely tunes in an anti-inflammatory modality the upstream mechanisms that are involved in the inflammatory cascade.

This mechanism is likely due to the ability of metaxalone to inhibit the augmented expression and activity of MAO-A. Indeed, blockade of this enzyme has been shown to cause marked antioxidant and anti-inflammatory activity. (See, Thangaleela, S. et al., Inhibition of monoamine oxidase attenuates social defeat-induced memory impairment in goldfish (Corassius auratus): A possible involvement of synaptic proteins and BDNF. Comp. Biochem. Physiol. C Toxicol. Pharmacol. 2021, 239, 108873)(See also, Liu, W. et al., Anti-inflammatory and protective effects of MT-031, a novel multitarget MAO-A and AChE/BuChE inhibitor in scopolamine mouse model and inflammatory cells. Neuropharmacology 2017, 113, 445-456).

Accordingly, our results have shown that IL-1β stimulation causes an increase in oxidative stress in HMC3 cells. Metaxalone treatment significantly reduced oxidative stress markers (MDA) and upregulated the mRNA expression and protein levels of NRF-2, chief regulator of the antioxidant system, when compared to cell cultures challenged with IL-1β alone.

The working examples herein demonstrate that metaxalone has important antioxidant and anti-inflammatory effects; in addition, metaxalone is currently on the market and severe side effects have not been reported. Therefore, it could be used for the management of neuroinflammation and pain, even in patients affected by fibromyalgia.

### Materials and Methods

### Cell Treatment

Human microglial HMC3 cell line (CRL-3304) was obtained from ATCC (American Type Culture Collection; Manassas, VA, USA). Cells were cultured in Eagle's minimum essential medium (EMEM) with a supplement of 10% fetal bovine serum (FBS) and 1% antibiotic mixture, and were incubated at 37° C. with 5% of CO2. Both medium and supplements were provided by ATCC (Manassas, VA, USA). Cells were seeded in 6-well plates at a density of 4×105 cells/well. A stock solution of metaxalone (Sigma Aldrich, Milan, Italy) was prepared by dissolving metaxalone in DMSO. Sixteen hours after plating (time 0), metaxalone was added at the doses of 10, 20, and 40 μg/mL 1 h following IL-1β stimulation (5 ng/mL; Sigma Aldrich, Milan, Italy). After 6 h cells were harvested for mRNA evaluation. In a separate set of plates, cells were incubated for 24 h before MTT assay to evaluate cell viability. The dose-response curve for IL-1β stimulation can be found at Supplemental FIG. S1.

### MTT Assay.

MTT assay was used to evaluate cell viability. HMC3 cells were grown and incubated with IL-1β (5 ng/mL). Cells were treated with different concentrations of metaxalone (10, 20, and 40 μg/mL) in a 96-well plate at a density of 4×104 cells/well for 24, 48, and 72 h to evaluate the cytotoxic effect of the drug; an additional group of cells was treated with staurosporine (100 nM; Sigma Aldrich, Milan, Italy) as a positive control. A mixture, constituted of 20 μL of the tetrazolium dye MTT 3-(4,5-dimethylthiazol-2-yl)-2,5-diphenyltetrazolium bromide (Sigma Aldrich, Milan, Italy), dissolved in sterile and filtered PBS, was added into each well 5 h before the end of the 24 h of incubation. Medium was then removed and the insoluble formazan crystals were dissolved through dimethyl sulfoxide (DMSO; 200 μL/well) addition, after 5 h. The difference in the values obtained at 540 and 620 nm of absorbance was used to evaluate the possible cytotoxic effect of metaxalone. The average of replicates was used for each group and the results were expressed as % of cell viability compared to untreated cells and reported as means and standard deviations.

### Real-Time Quantitative PCR Amplification (RtqPCR).

Total RNA was extracted from human microglial HMC3 for RT-qPCR using Trizol LS reagent (Invitrogen, Carlsbad, CA, USA). Two μg of total RNA was reverse transcribed in a final volume of 20 μL using a Superscript VILO kit (Invitrogen, Carlsbad, CA, USA). The obtained cDNA (1 μL) was added to the EvaGreen qPCR Master Mix (Biotium Inc., Fremont, CA, USA), achieving a final volume of 20 μL per well. The final primer concentration used to carry out the analysis was 10 μM. Samples from each group were run in duplicate and β-actin was used as an endogenous control. Results were calculated using the 2-ΔΔCT method and expressed as n-fold increase in gene expression using control group as calibrator. Irrera, N. et al. Activation of A2A Receptor by PDRN Reduces Neuronal Damage and Stimulates WNT/β-CATENIN Driven Neurogenesis in Spinal Cord Injury. Front. Pharmacol. 2018, 9, 506. Pizzino, G. et al. Effects of the antagomiRs 15b and 200b on the altered healing pattern of diabetic mice. Br. J. Pharmacol. 2018, 175, 644-655.

Measurements of Cytokines by Enzyme-Linked Immunosorbent Assay (ELISA). Tumor necrosis factor-alpha (TNF-α), interleukin-6 (IL-6) and interleukin-13 (IL-13) were measured in the cell culture supernatants. The products under investigation were measured using enzyme-linked immunosorbent assay (ELISA) kits (Abcam, Cambridge, UK), in agreement with the instructions reported by the manufacturer. All the samples were evaluated in duplicate and the obtained results were interpolated with the pertinent standard curves. To evaluate the sample, the means of the duplicated sample were used and expressed in pg/mL.

MAO-A Activity Assay. MAO-A activity was evaluated by a two-step bioluminescent assay methodology, as previously described (See, Lin, Y. C. et al., MAO-A novel decision maker of apoptosis and autophagy in hormone refractory neuroendocrine prostate cancer cells. Sci. Rep. 2017, 7, 46338) using a MAO-GLO™ assay kit from Promega (Madison, WI, USA). The produced amount of signal (light intensity) was measured using a microplate luminometer (Multilabel Counter Victor3™, PerkinElmer Life Sciences). Human recombinant MAO-A enzyme was used as a positive control. The results are given as relative light units after the background was subtracted.

Malonaldehyde Assay. Metaxalone antioxidant effects were examined in HMC3 cells by measuring malonaldehyde (MDA) levels as a marker of lipid peroxidation, as previously described in detail. (See, Ceravolo, I. Et al., Health Potential of Aloe vera against Oxidative Stress Induced Corneal Damage: An “In Vitro” Study. Antioxidants 2021, 10, 318).

Western Blot Analysis. HMC3 cells were homogenized in RIPA buffer (25 mM Tris/HCl, pH 7.4; 1.0 mM EGTA; and 1.0 mM EDTA) with 1% of NP40, 0.5% of phenyl methylsulfonyl fluoride (PMSF), aprotinin, and leupeptin and pepstatin (10 μg/mL each) to perform protein extraction. Lysates were centrifuged at 15,000×g for 15 min at 4° C. and the supernatant was collected for protein determination using a specific kit (Bio-Rad DC; Bio-Rad, Richmond, CA, USA). Samples were denatured in a reducing buffer (62 mM Tris pH 6.8, 10% glycerol, 2% SDS, 5% β-mercaptoethanol, and 0.003% bromophenol blue) and proteins were separated by electrophoresis on SDS polyacrylamide gels.

Following electrophoresis, samples were transferred onto a PVDF membrane (Amersham, Little Chalfont, UK) in a transfer buffer (39 mM glycine, 48 mM Tris pH 8.3, and 20% methanol) at 200 mA for 1 h. The obtained membranes were incubated with 5% non-fat dry milk in TBS/0.1% Tween for 1 h at room temperature, washed 3 times in TBS/0.1% Tween, and incubated with primary antibodies to detect pNF-κB, PPARγ, PGC-1α (Cell Signaling, Danvers, MA, USA), NRF-2, and MAO-A (Abcam, Cambridge, UK), diluted in TBS/0.1% Tween overnight at 4° C. The day after, following 3 washes with TBS/0.1% Tween, membranes were incubated with secondary peroxidase-conjugated goat anti-mouse and anti-rabbit antibodies (KPL, Gaithersburg, MD, USA) for 1 h at room temperature. After washing, membranes were analyzed by the enhanced chemiluminescence system (LumiGlo reserve; Seracare, Milford, MA, USA). The protein signal was detected and quantified by scanning densitometry using a bio-image analysis system (C-DiGit, Li-cor, Lincoln, NE, USA). The results were expressed as relative integrated intensity and β-actin (Cell Signaling, Danvers, MA, USA) was used to confirm equal protein loading. (See, Irrera, N. et al. β-Caryophyllene Inhibits Cell Proliferation through a Direct Modulation of CB2 Receptors in Glioblastoma Cells. Cancers 2020, 12, 1038).

Statistical Analysis. Data are shown as mean±SD and the reported values are the result of at least five experiments. To ensure reproducibility, all assays were replicated three times. The various groups were compared and evaluated using one-way ANOVA with Tukey post-test for comparison between the different groups. A p value <0.05 was considered significant. Graphs were created and organized using GraphPad Prism (version 8.0 for macOS, San Diego, CA, USA).

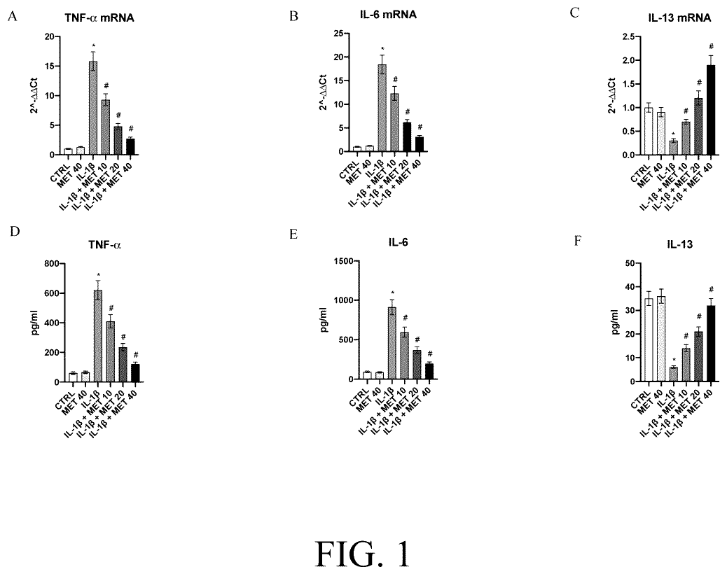

Results. Metaxalone Reverts the Inflammatory Phenotype Induced by IL-1β in HMC3 Microglial Cells. Inflammatory cytokines released by microglial cells orchestrate an inflammatory cascade that induces the development and progression of a long-term neuroinflammation, which plays a key role in the development and perpetuation of several neurological disorders, including fibromyalgia. For this reason, metaxalone's effects on reducing an inflammatory phenotype were evaluated after stimulating HMC3 microglial cells with IL-1β. IL-1β challenge caused a significant increase in the expression of pro-inflammatory cytokines TNF-α and IL-6 (p<0.0001 vs. CTRL) and a decrease in the anti-inflammatory cytokine IL-13 (p<0.0001 vs. CTRL) expression, evaluated either as mRNA expression or protein levels (See, FIG. 1). Metaxalone significantly reverted the inflammatory phenotype prompted by IL-1β in a dose-dependent manner (p<0.0001 vs. IL-1β; see, FIG. 1).

Ijms 22 08425 g001 550, see, FIG. 1. Effects of metaxalone on TNF-α (A), IL-6 (B), and IL-13 (C) mRNA expression. Effects of metaxalone on TNF-α (D), IL-6 (E), and IL-13 (F) protein levels. Values are expressed as the means±SD. *, p<0.0001 vs. CTRL; #, p<0.0001 vs. IL-1β.

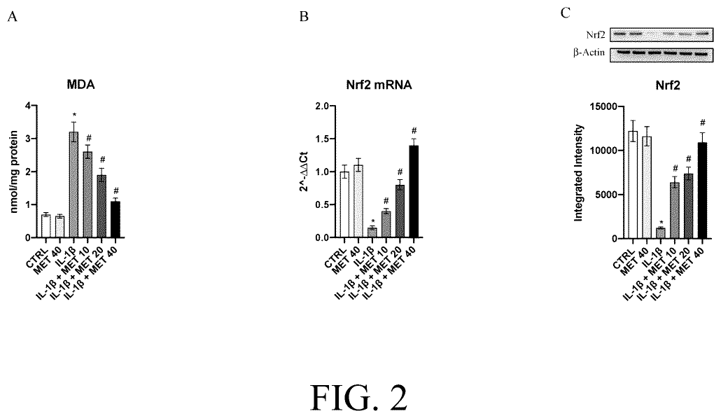

Effects of Metaxalone on Oxidative Stress. A significant decrease in NRF-2 mRNA expression was observed in IL-1β-challenged cells compared to the control group, as a consequence of oxidative stress induction following IL-1β stimulation (p<0.0001 vs. CTRL; see FIG. 2). Metaxalone treatment significantly upregulated NRF-2 gene expression in HMC3 cells (p<0.0001 vs. IL-1β; see, FIG. 2). Moreover, NRF-2 mature protein levels were significantly reduced in the IL-1β group compared to the control group (p<0.0001 vs. CTRL; see, FIG. 2). Furthermore, metaxalone treatment significantly increased NRF-2 levels in HMC3 cells after exposure to IL-1β stimulus in a dose-dependent manner (p<0.0001 vs. IL-1β; see, FIG. 2).

Ijms 22 08425 g002 550. See, FIG. 2. Effects of metaxalone on MDA generation (A), NRF-2 mRNA expression (B), and NRF-2 protein levels (C). Values are expressed as the means±SD. *, p<0.0001 vs. CTRL; #, p<0.0001 vs. IL-1β.

MDA levels were measured in the HMC3 cells to better characterize metaxalone antioxidant effects. Control cells showed low levels of MDA whereas IL-1β stimulation considerably increased MDA levels (p<0.0001 vs. CTRL; see, FIG. 2). Metaxalone treatment caused a significant reduction in MDA levels in HMC3 cells (p<0.0001 vs. IL-1β; see, FIG. 2), thus confirming its antioxidant properties.

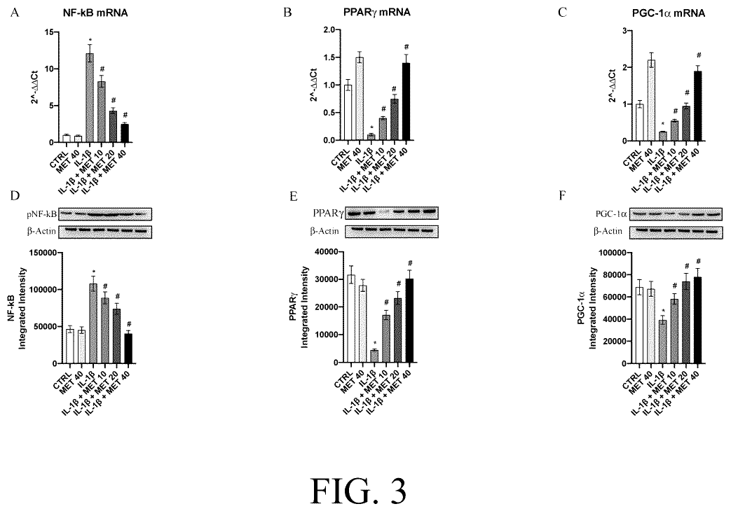

Metaxalone Targets Upstream Signals That Trigger the Inflammatory Phenotype. The transcription factor NF-κB was markedly induced by IL-1β challenge in HMC3 cells (p<0.0001 vs. CTRL; see, FIG. 3); metaxalone significantly reduced its mRNA expression in a dose-dependent manner (p<0.0001 vs. IL-1p; see, FIG. 3). In addition, IL-1β stimulation also suppressed PPARγ and PGC-1α expression in microglial cells (p<0.0001 vs. CTRL; see, FIG. 3), and metaxalone was able to trigger a marked increase in the expression of both the nuclear receptor and its co-activator when compared to cell cultures challenged with IL-1β (p<0.0001 vs. IL-1β; see, FIG. 3).

Ijms 22 08425 g003 550. See, FIG. 3. Effects of metaxalone on NF-kB (A), PPARγ (B), and PGC-1α (C) mRNA expression. Effects of metaxalone on pNF-kB (D), PPARγ (E), and PGC-1α (F) protein levels. Values are expressed as the means±SD. *, p<0.0001 vs. CTRL; #, p<0.0001 vs. IL-1β.

The mature protein levels of inflammatory markers were measured in HMC3 cells stimulated with IL-1β to confirm metaxalone's anti-inflammatory effects. p-NF-κB expression was markedly increased following IL-1β stimulus in HMC3 cells (p<0.0001 vs. CTRL; see, FIG. 3) and, by contrast, metaxalone treatment blunted the increase in p-NF-κB protein (p<0.0001 vs. IL-1β; see, FIG. 3). IL-1β challenge also significantly decreased PPARγ and PGC-1α protein expression in HMC3 cells compared to controls (p<0.0001 vs. CTRL; see, FIG. 3), whereas metaxalone treatment stimulated PPARγ and PGC-1α activation when compared to cell cultures challenged with IL-1β (p<0.0001 vs. IL-1β; see, FIG. 3), as demonstrated by their protein expression.

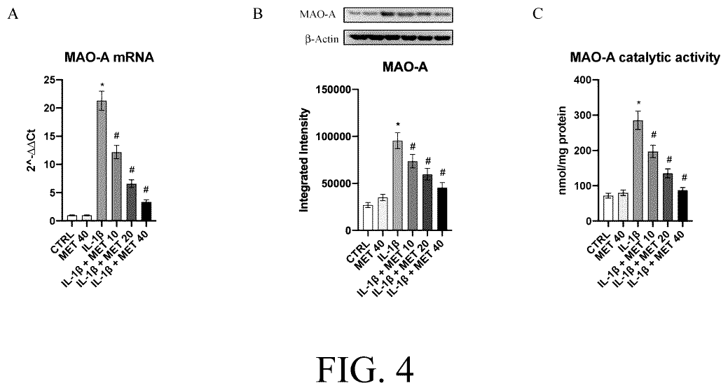

Metaxalone Reduces the Augmented MAO-A Expression and Activity Induced by IL-1β in Microglia Cells. MAO-A was constitutively expressed in microglia cells (See, FIG. 4). IL-1β challenge caused a robust increase in MAO-A mRNA expression and protein levels (p<0.0001 vs. CTRL; see, FIG. 4A,B); metaxalone markedly reduced MAO-A mRNA expression in a dose-dependent fashion (p<0.0001 vs. IL-1β; FIG. 4A,B). MAO-A activity was also studied by using a chemiluminescence assay: IL-1β incubation resulted in an increased activity of MAO-A (p<0.0001 vs. CTRL; see, FIG. 4C). Thus, under our experimental condition, the triggering of an inflammatory phenotype in microglial cells was accompanied by an increase in MAO-A expression and, more interestingly, activity. As expected, metaxalone also suppressed MAO-A activity in a dose-dependent manner (p<0.0001 vs. IL-1β; see, FIG. 4C).

Ijms 22 08425 g004 550. See, FIG. 4. Effects of metaxalone on MAO-A mRNA expression (A), MAO-A protein levels (B), MAO-A activity (C). Values are expressed as the means±SD. *, p<0.0001 vs. CTRL; #, p<0.0001 vs. IL1β.

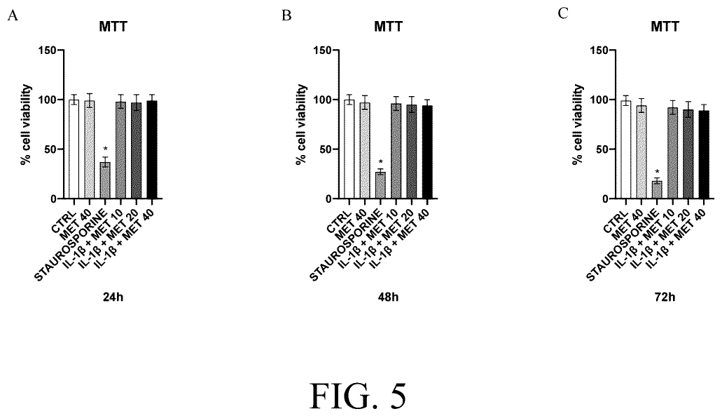

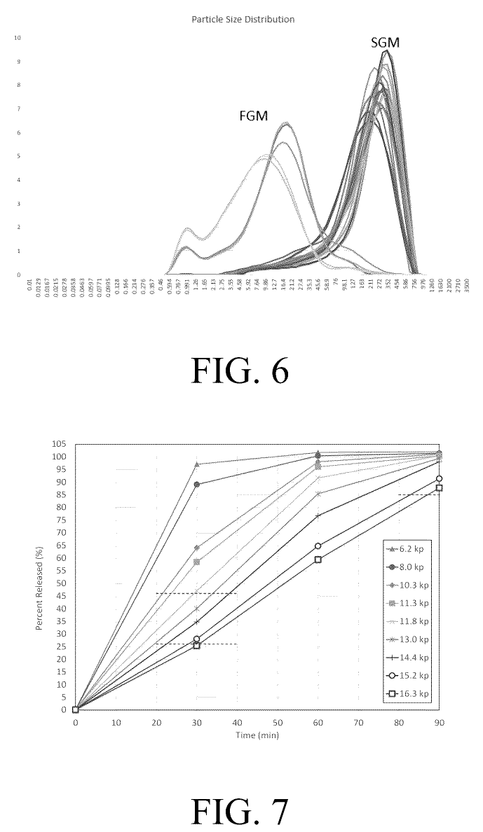

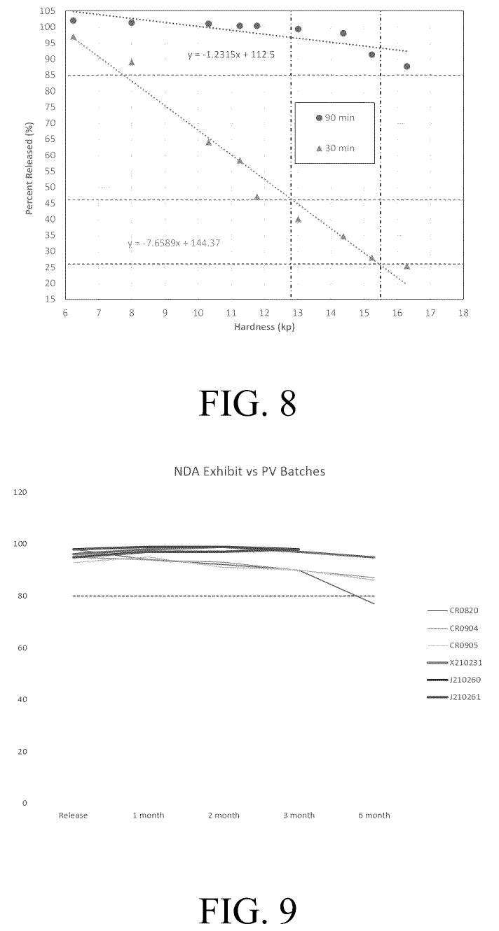

Effect of Metaxalone on HMC3 Microglial Cells Viability. An MTT test was performed to evaluate the possible occurrence of a toxic effect following metaxalone treatment in microglial cells. Specifically, cells were incubated for 24, 48, and 72 h with the same concentrations of metaxalone, 10, 20, and 40 μg/mL. As shown in FIG. 5, metaxalone did not produce any toxic effect in the treated cells and related viability percentage kept similar to control at all reported concentrations and time points, whereas the staurosporine-treated group (positive control) showed a significant reduction in cell viability at 24, 48, and 72 h (See, FIG. 5).

Ijms 22 08425 g005 550. See, FIG. 5. Effects of metaxalone on cell viability at 24 h (A), 48 h (B), and 72 h (C). Values are expressed as the means±SD. *, p<0.0001 vs. CTRL.

### Example 2

Table 1 describes a representative batch formulation for a 640 mg tablet of the current invention:

TABLE 1

| | Quantity/Batch | |
|---|---|---|
| Ingredient | (kg) | |
| Metaxalone First Grade (FGM) | 53.760 | kg |
| Metaxalone Second Grade (SGM) | 35.840 | kg |
| Lactose Monohydrate | 3.764 | kg |
| FD&C Yellow #6 | 0.045 | kg |
| Propylene Glycol Alginate | 2.240 | kg |
| Alginic Acid | 2.240 | kg |
| Povidone | 6.720 | kg |
| Purified Water* | 22.8 | kg |
| Magnesium Stearate | 0.672 | kg |
| Total | 105.28 | kg |

*Not present in final product.

Distributions of particles sizes from representative batches of first grade and regular metaxalone are provided in FIG. 6.

### Example 3

This example includes detailed information describing the manner in which Metaxalone Tablets 640 mg are manufactured, using the formulation and metaxalone described in Table 1.

Granulating Solution: Povidone was dissolved by adding to Purified Water while mixing using mixer at required speed.

Pre-Mixing: Metaxalone First Grade, Metaxalone Second Grade, FD&C Yellow #6, Propylene Glycol Alginate, and Alginic Acid were mixed at a suitable head speed.

Wet-Granulation: To the above Pre-Mix blend, granulating solution was added while mixing at suitable head speed.

Drying: Wet-Granulation was dried in an oven dryer to achieve desired moisture content.

Milling: Upon completion of drying process, dried granulation was milled using comminuting mill.

Final-Mixing: Magnesium Stearate was added to milled blend and lubricated using suitable blender.

Compression: Final-Mix Blend was compressed into tablets using Rotary Tablet Press.

### Example 4

Dissolution was subsequently tested for a single granulate compressed at a range of hardness values, ranging from 6.2 to 16.3 kp. All samples were tested according to USP Metaxalone Tablets monograph and the dissolution is shown in FIG. 7. Dissolution versus hardness is plotted in FIG. 8. The hardness limits were obtained by this method with the minimum value set for the upper hardness limit comparing the 30 and 90 minute trendlines, 12.5 and 15.2 kp. The hardness target was set as the midpoint of the upper and lower specification, 13.9 kp, and the action limits were set at +/−1.0 kp from the target, 12.9 and 14.9 kp. The remainder of the batch was then compressed on a Rotary Tablet Press, at the target hardness.

### Example 5

In order to determine the improvement provided by the manufacturing processes disclosed herein, stability testing was performed to determine the stability of the dissolution profile of tablets manufacturing according to the methods described herein, compared to the tablets produced by the methods described in PCT/US2020/039041, each having an identical qualitative/quantitative formulation. In particular, tablets from three batches of product manufactured according to PCT/US2020/039041 (“NDA exhibit” batches) and tablets from three batches manufactured according to the claimed invention (“process validation” or “PV” batches), were stored in a stability chamber at ICH Q1A (R2) accelerated conditions (40±2° C./75%±5% relative humidity) to simulate product shelf life and tested periodically for dissolution kinetics according to USP<711> Dissolution method for the USP Metaxalone Tablets monograph Test 3 acceptance criteria, at 30 minute (PV Batch only) and 90 minute (PV and NDA Exhibit Batches) time points. 90-minute dissolution results are depicted in FIG. 9 and tabulated in Table 2:

TABLE 2
% Dissolution

| | | | | |
|---|---|---|---|---|
| Product | | | | |
| Release | 1 month | 2 month | 3 month | 6 month |

NDA Exhibit

| | | | | | |
|---|---|---|---|---|---|
| CR0820 | 98 | 94 | 92 | 90 | 77 |
| CR0904 | 95 | 94 | 93 | 90 | 87 |
| CR0905 | 93 | 95 | 91 | 90 | 86 |

PV Batches

| | | | | | |
|---|---|---|---|---|---|
| X210231 | 96 | 98 | 99 | 97 | 95 |
| J210260 | 98 | 99 | 99 | 98 | 95 |
| J210261 | 95 | 97 | 97 | 98 | 92 |

A can be seen, the three NDA exhibit batches did not maintain their dissolution stability over time. This is in stark contrast to the three PV batches, which did.

1. A method of treating chronic or acute conditions associated with specific or non-specific neuroinflammation in a human consisting of inhibiting the MAO-A isoform enzymatic pathway by administering an oral dosage form comprising:
a therapeutic dose of first and second particulate metaxalones in one or more suitable forms selected from salts, co-crystals, solvates, free form, enantiomers, and polymorphs thereof, and,
one or more pharmaceutically suitable excipients, wherein the dosage form has a stable dissolution profile over time.

2. (canceled)

3. (canceled)

4. A method of restoring or increasing IL-13 and inhibiting STAT3 consisting of administering an oral dosage form comprising:
a therapeutic dose of first and second particulate metaxalones in one or more suitable forms selected from salts, co-crystals, solvates, free form, enantiomers, and polymorphs thereof, and,
one or more pharmaceutically suitable excipients, wherein the dosage form has a stable dissolution profile over time.

5. The method of claim 4, further consisting of increasing white matter growth and development in the central nervous system by increasing and/or attenuating the natural pathways responsible for proper white matter cellular growth and repair.

6. The method of claim 5, further consisting of treating demyelination disorders or diseases.

7. A method of increasing or restoring PGC-1α consisting of administering an oral dosage form comprising:
a therapeutic dose of first and second particulate metaxalones in one or more suitable forms selected from salts, co-crystals, solvates, free form, enantiomers, and polymorphs thereof, and,
one or more pharmaceutically suitable excipients, wherein the dosage form has a stable dissolution profile over time.

8. The method of claim 7, further consisting of treating one or more disorders or diseases selected from sciatica, fibromyalgia, neuropathic pain, ischemic stroke and traumatic brain injury.

9. The method of claim 8, wherein said neuropathic pain is selected from mechanical allodynia, thermal hyperalgesia, diabetic neuropathy, chemotherapy neuropathy and post-operative pain.

10. The method of claim 7, further consisting of restoring mitochondrial biogenesis.

11. The method of claim 7, further consisting of stimulating NRF-1 and NRF-2.

12. The method of claim 7, further consisting of preventing chronification of neuropathic pain due to haploinsufficiency.

13. The method of claim 7, further consisting of stimulating AMPK.

14. The method of claim 13, further consisting of bolstering gene expression and protein syntheses of MnSOD and catalase.

15. The method of claim 13, further consisting of treating cardiovascular disorder or disease and treating vascular leakage caused by permeability.

16. The method of claim 7, further consisting of treating pain caused by nerve injury.

17. The method of claim 7, further consisting of preventing pain chronification caused by burn injury.

18. (canceled)

19. The method of claim 7, further consisting of inhibiting NF-κB.

20. The method of claim 7, further consisting of revitalizing muscle strength.

21. The method of claim 7, further consisting of initiating regeneration of muscular tissue.

22. The method of claim 7, further consisting of promoting an antifibrotic state.

23-47. (canceled)
